(19) United States
(12) Patent Application Publication
(10) Pub. No.: US 2008/0075704 A1
(43) Pub. Date: Mar. 27, 2008

(54) Method of providing readily available cellular material derived from peripheral blood, and a composition thereof

(76) Inventors: David A. Wolf, Houston, TX (US); Donnie Rudd, Sugar Land, TX (US)

Correspondence Address: LADAS & PARRY LLP, 224 SOUTH MICHIGAN AVENUE, SUITE 1600, CHICAGO, IL, 60604, US

(21) Appl. No.: 11/894,098

(22) Filed: Aug. 20, 2007

Related U.S. Application Data
(63) Continuation-in-part of application No. 11/363,702, filed on Feb. 27, 2006.
(60) Provisional application No. 60/657,306, filed on Feb. 28, 2005.
(60) Provisional application No. 60/657,271, filed on Feb. 28, 2005.

(51) Int. Cl. A01N 1/02; A61K 35/00; A61P 43/00; C12N 13/00; C12N 15/01; C12N 5/06; C12Q 1/02

(52) U.S. Cl. 424/93.21; 435/173.1; 435/2; 435/29; 435/372; 435/446

(57) ABSTRACT

The present invention is directed to the process of preparing an expanded peripheral blood stem cell composition, preparing a TVEMF-expanded peripheral blood stem cell composition, methods of producing the same, and to repair of tissue and/or function with expanded, and TVEMF-expanded, peripheral blood stem cells.

### FIELD OF THE INVENTION

The present invention is directed to adult stem cells from peripheral blood prepared in a TVEMF-bioreactor, and to a process for such preparation, compositions thereof, and methods of treating a mammal with the cells or compositions.

### BACKGROUND OF THE INVENTION

Regeneration of mammalian, particularly human, tissue has long been a desire of the medical community. Thus far, repair of human tissue has been accomplished largely by transplantations of like tissue from a donor. Beginning essentially with the kidney transplant from one of the Herrick twins to the other and later made world famous by South African Doctor Christian Barnard's transplant of a heart from Denise Darval to Louis Washkansky on Dec. 3, 1967, tissue transplantation became a widely accepted method of extending life in terminal patients.

Transplantation of human tissue, from its first use, encountered major problems, primarily tissue rejection due to the body's natural immune system. This often caused the use of tissue transplantation to have a limited prolongation of life (Washkansky lived only 18 days past the surgery).

In order to overcome the problem of the body's immune system, numerous anti-rejection drugs (e.g. Imuran, Cyclosporine) were soon developed to suppress the immune system and thus prolong the use of the tissue prior to rejection. However, the rejection problem has continued creating the need for an alternative to tissue transplantation.

Bone marrow transplantation has also been used, and is still the procedure of choice for treatment of some illnesses, such as leukemia, to repair certain tissues such as bone marrow, but bone marrow transplantation also has problems. It requires a match from a donor (found less than 50% of the time); it is painful, expensive, and risky. Consequently, an alternative to bone marrow transplantation is highly desirable. Transplantation of tissue stem cells such as the transplantation of liver stem cells found in U.S. Pat. No. 6,129,911 have similar limitations rendering their widespread use questionable.

In recent years, researchers have experimented with the use of pluripotent embryonic stem cells as an alternative to tissue transplant. The theory behind the use of embryonic stem cells has been that they can theoretically be utilized to regenerate virtually any tissue in the body. The use of embryonic stem cells for tissue regeneration, however, has also encountered problems. Among the more serious of these problems are that transplanted embryonic stem cells have limited controllability, they sometimes grow into tumors, and the human embryonic stem cells that are available for research would be rejected by a patient's immune system (Nature, Jun. 17, 2002: Pearson, “Stem Cell Hopes Double”, news@nature.com, published online:21 Jun. 2002). Further, widespread use of embryonic stem cells is so burdened with ethical, moral, and political concerns that its widespread use remains questionable.

The pluripotent nature of stem cells was first discovered from an adult stem cell found in bone marrow. Verfaille, C. M. et al., Pluripotency of mesenchymal stem cells derived from adult marrow. Nature 417, published online 20 June; doi: 10.1038/nature00900, (2002) cited by Pearson, H. Stem cell hopes double. news@nature.com, published online:21 Jun. 2002; doi: 10.1038/news020617-11.

Boyse et al., U.S. Pat. No. 6,569,427 B1, discloses the cryopreservation and usefulness of cryopreserved fetal or neonatal blood in the treatment or prevention of various diseases and disorders such as anemias, malignancies, autoimmune disorders, and various immune dysfunctions and deficiencies. Boyse also discloses the use of hematopoietic reconstitution in gene therapy with the use of a heterologous gene sequence. The Boyse disclosure stops short, however, of expansion of cells for therapeutic uses. C or Cell, a cord blood bank, provides statistics on expansion, cryopreservation, and transplantation of umbilical cord blood stem cells. “Expansion of Umbilical Cord Blood Stem Cells”, Information Sheet Umbilical Cord Blood, CorCell, Inc. (2003). One expansion process discloses utilizing a bioreactor with a central collagen based matrix. Research Center Julich: Blood Stem Cells from the Bioreactor. Press release May 17, 2001.

Research continues in an effort to elucidate the molecular mechanisms involved in the expansion of stem cells. For example, the C or Cell article discloses that a signal molecule named Delta-1 aids in the development of cord blood stem cells. Ohishi K. et al.: Delta-1 enhances marrow and thymus repopulating ability of human CD34+/CD38− cord blood cells. Clin. Invest. 110:1165-1174 (2002).

Throughout this application, the term “peripheral blood” means blood that circulates, or has circulated, systematically in a mammal. The term “peripheral blood cells” means cells found in peripheral blood.

While adult stem cells can be found in numerous mature tissues, they are found in lesser quantities and are harder to locate. Also, stem cells found in tissues may be dedicated to that tissue, and less able to function as a truly pluripotent cell. Peripheral blood cells, however, are more readily available than stem cells in tissues.

There is a need, therefore, to provide a method and process of repairing human tissue that is not based on organ transplantation, bone marrow transplantation, or embryonic stem cells, and yet provides a composition of expanded peripheral blood stem cells, preferably in a therapeutic condition and dosage and unlikely to elicit an immune response, for use in a matter of hours rather than days.

### SUMMARY OF THE INVENTION

The present invention relates in part to peripheral blood stem cells from a mammal, preferably human, preferably wherein said stem cells are TVEMF-expanded. The present invention also relates to peripheral blood stem cells from a mammal, preferably human, wherein said stem cells are expanded in a rotating bioreactor, preferably to at least seven times greater than the number before expansion. The present invention also relates to introducing to the mammal a therapeutically effective amount of expanded adult stem cells, or TVEMF-expanded adult stem cells repair tissue and/or tissue function. The invention also relates to compositions comprising these cells, with other components added as desired, including pharmaceutically acceptable carriers, cryopreservatives, and cell culture media.

The present invention also relates to a process for preparing expanded, preferably TVEMF-expanded, peripheral blood stem cells and stem cell compositions for repairing tissue and/or function by placing a peripheral blood mixture in a culture chamber of a rotatable bioreactor, preferably a TVEMF-bioreactor; and rotating the bioreactor. In a preferred embodiment, the rotating bioreactor is a TVEMF bioreactor which is provided with the additional step of subjecting the peripheral blood stem cell mixture to a TVEMF and TVEMF-expanding the peripheral blood stem cells in the TVEMF bioreactor to prepare TVEMF-expanded peripheral blood stem cells and a stem cell composition. The present invention also relates to a method of cryopreserving the expanded stem cells by lowering their temperature to −120° C. to −196° C. for one year or longer.

### BRIEF DESCRIPTION OF THE DRAWINGS

In the drawings,

FIG. 1 schematically illustrates a preferred embodiment of a culture carrier flow loop of a bioreactor;

FIG. 2 is an elevated side view of a preferred embodiment of a TVEMF-bioreactor of the invention;

FIG. 3 is a side perspective of a preferred embodiment of the TVEMF-bioreactor of FIG. 2;

FIG. 4 is a vertical cross sectional view of a preferred embodiment of a TVEMF-bioreactor;

FIG. 5 is a vertical cross sectional view of a TVEMF-bioreactor;

FIG. 6 is an elevated side view of a time varying electromagnetic force source that can house, and provide a time varying electromagnetic force to, a bioreactor;

FIG. 7 is a front view of the TVEMF source shown in FIG. 6;

FIG. 8 is a front view of the TVEMF source shown in FIG. 6, further showing a bioreactor therein,

FIG. 9 is the orbital path of a typical cell in a non-rotating reference frame;

FIG. 10 is a graph of the magnitude of deviation of a cell per revolution;

FIG. 11 is a representative cell path as observed in a rotating reference frame of the culture medium;

FIG. 12 illustrates the expansion pattern of total nucleated cells in a rotating bioreactor versus a dynamic moving culture;

FIG. 13 illustrates the expansion pattern of CD133+ cells in a rotating bioreactor versus a dynamic moving culture;

FIG. 14 illustrates the expansion pattern of CD34+ cells in a rotating bioreactor versus a dynamic moving culture;

FIG. 15 is a graphic illustration of the expansion (increase in number) from day 0 to day 6 of CD34+ cells cultured in a rotating TVEMF-bioreactor; and

FIG. 16 illustrates the number of CD34+ cells at day 6 in a TVEMF-expansion culture as compared with and a non-TVEMF expansion culture.

### DETAILED DESCRIPTION OF THE DRAWINGS

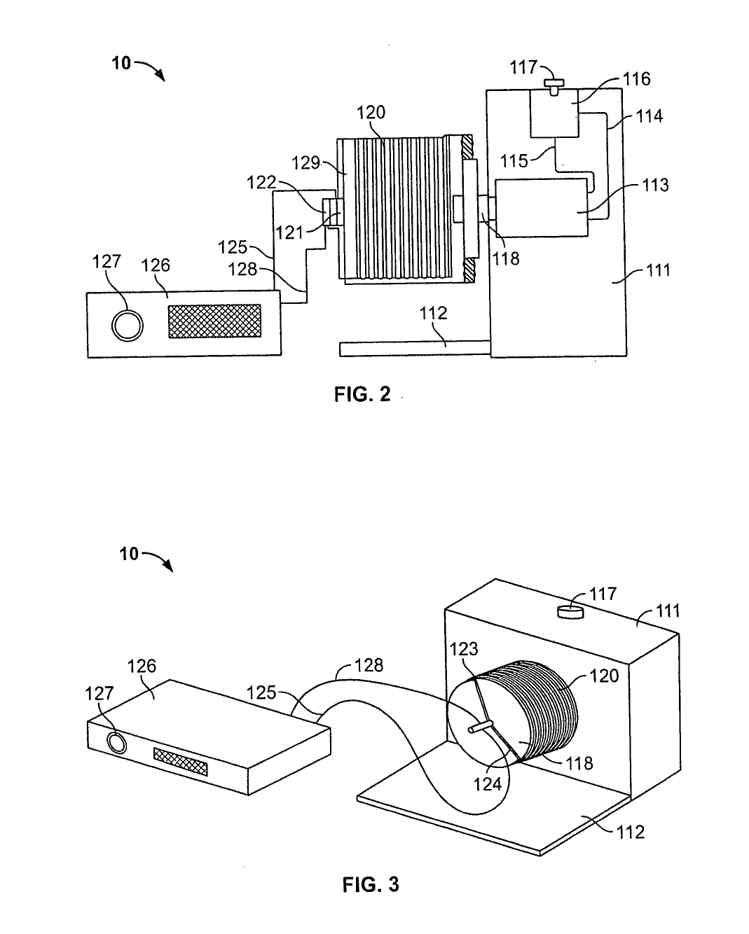

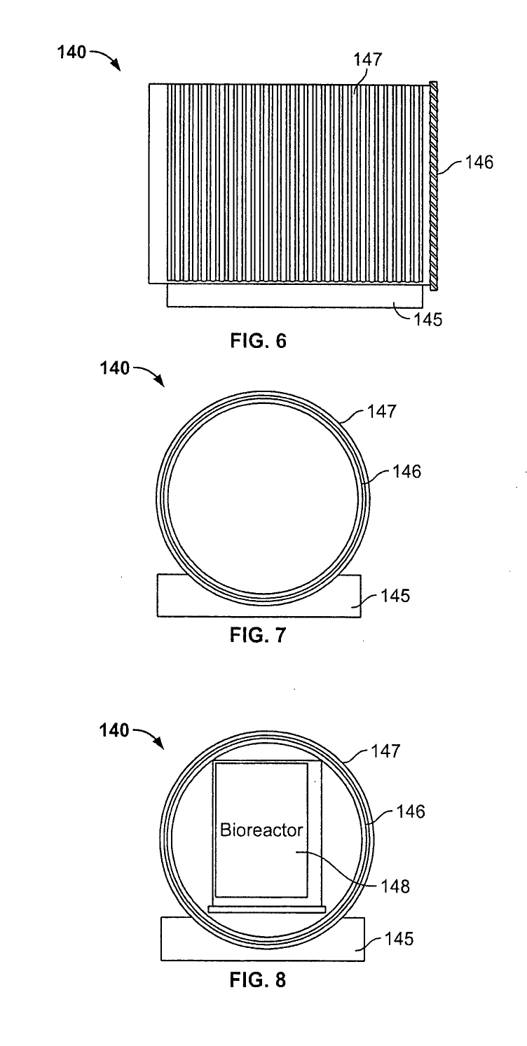

In the simplest terms, a rotating bioreactor comprises a cell culture chamber and a time varying electromagnetic force source. In operation, a blood mixture is placed into the cell culture chamber. The cell culture chamber is filled so as to create a three-dimensional environment wherein each individual non-adherent peripheral blood cell is suspended. The cell culture chamber is rotated in one direction, 360 degrees, over a period of time during which a time varying electromagnetic force is generated in the chamber by the time varying electromagnetic force source. During their time in the rotating bioreactor, the cells are suspended in discrete microenvironments in the essentially quiescent three-dimensional environment created therein. In a preferred embodiment, the rotating bioreactor is a TVEMF-bioreactor wherein, in addition to being suspended by rotating, the cells are exposed to a time varying electromagnetic force to provide additional unique characteristics to the cells and to enhance the expansion process. Upon completion of the time, the expanded blood mixture is removed from the chamber. In a more complex TVEMF-bioreactor system, the time varying electromagnetic force source can be integral to the TVEMF-bioreactor, as illustrated in FIGS. 2-5, but can also be adjacent to a bioreactor as in FIGS. 6-8. The TVEMF source preferably comprises a TVEMF generating device, which may preferably be a coil, more preferably at least one loop. Furthermore, a fluid carrier such as cell culture media or buffer (preferably similar to that media added to a blood mixture, discussed below), which provides sustenance to the cells, can be periodically refreshed and removed. Preferred TVEMF-bioreactors are described herein. However, it is also contemplated that tissue and/or function can be repaired by using a non-TVEMF rotating bioreactor to expand peripheral blood stem cells.

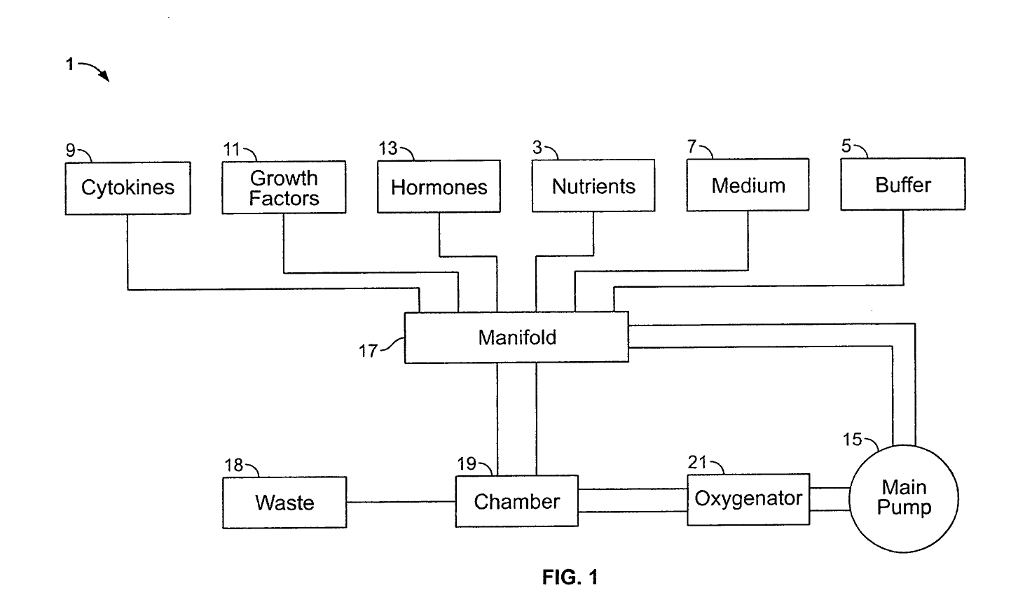

Referring now to FIG. 1, illustrated is a preferred embodiment of a culture carrier flow loop 1 in an overall bioreactor culture system for growing mammalian cells having a cell culture chamber 19, preferably a rotating cell culture chamber, an oxygenator 21, an apparatus for facilitating the directional flow of the culture carrier, preferably by the use of a main pump 15, and a supply manifold 17 for the selective input of such culture carrier requirements as, but not limited to, nutrients 3, buffers 5, fresh medium 7, cytokines 9, growth factors 11, and hormones 13. In this preferred embodiment, the main pump 15 provides fresh fluid carrier to the oxygenator 21 where the fluid carrier is oxygenated and passed through the cell culture chamber 19. The waste in the spent fluid carrier from the cell culture chamber 19 is removed and delivered to the waste 18 and the remaining cell culture carrier is returned to the manifold 17 where it receives a fresh charge, as necessary, before recycling by the pump 15 through the oxygenator 21 to the cell culture chamber 19.

In the culture carrier flow loop 1, the culture carrier is circulated through the living cell culture in the chamber 19 and around the culture carrier flow loop 1, as shown in FIG. 1. In this loop 1, adjustments are made in response to chemical sensors (not shown) that maintain constant conditions within the cell culture reactor chamber 19. Controlling carbon dioxide pressures and introducing acids or bases corrects pH. Oxygen, nitrogen, and carbon dioxide are dissolved in a gas exchange system (not shown) in order to support cell respiration. The closed loop 1 adds oxygen and removes carbon dioxide from a circulating gas capacitance. Although FIG. 1 is one preferred embodiment of a culture carrier flow loop that may be used in the present invention, the invention is not intended to be so limited. The input of culture carrier elements such as, but not limited to, oxygen, nutrients, buffers, fresh medium, cytokines, growth factors, and hormones into a bioreactor can also be performed manually, automatically, or by other control means, as can be the control and removal of waste and carbon dioxide.

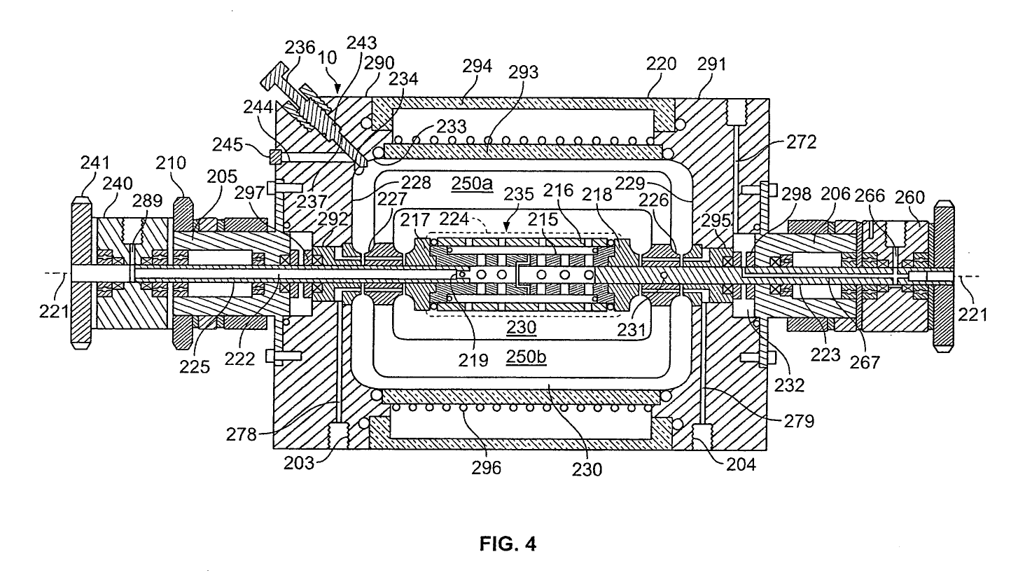

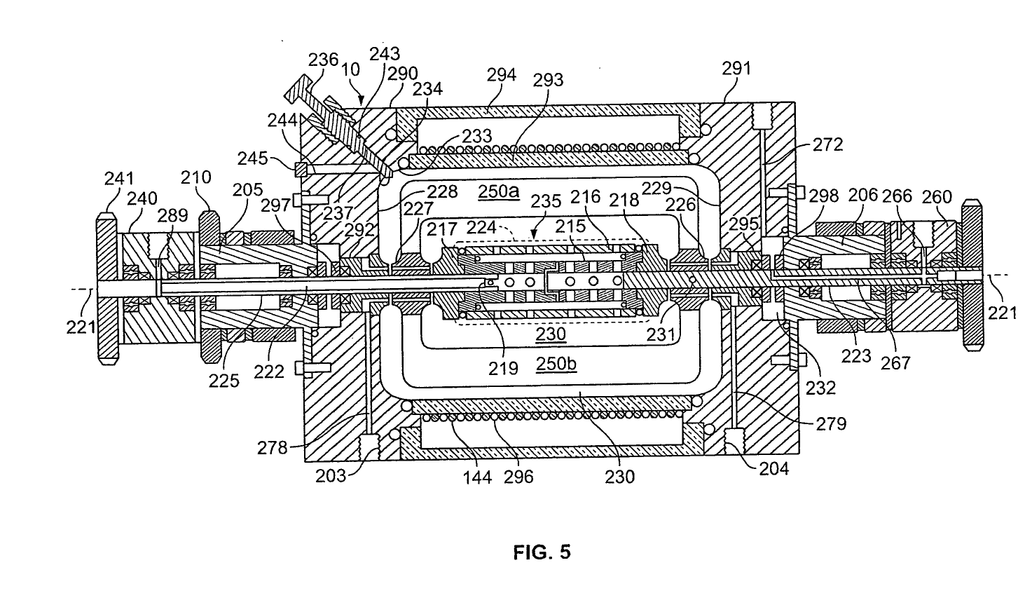

FIGS. 2 and 3 illustrate a preferred embodiment of a TVEMF-bioreactor 10 with an integral time varying electromagnetic force source. FIG. 4 is a cross section of a rotatable TVEMF-bioreactor 10 for use in the present invention in a preferred form. The TVEMF-bioreactor 10 of FIG. 4 is illustrated with an integral time varying electromagnetic force source. FIG. 5 also illustrates a preferred embodiment of a TVEMF-bioreactor with an integral time varying electromagnetic force source. FIGS. 6-8 show a rotating bioreactor with an adjacent time varying electromagnetic force source.

Turning now to FIG. 2, illustrated in FIG. 2 is an elevated side view of a preferred embodiment of a TVEMF-bioreactor 10 of the present invention. FIG. 2 comprises a motor housing 111 supported by a base 112. A motor 113 is attached inside the motor housing 111 and connected by a first wire 114 and a second wire 115 to a control box 116 that has a control means therein whereby the speed of the motor 113 can be incrementally controlled by turning the control knob 117. The motor housing 111 has a motor 113 inside set so that a motor shaft 118 extends through the housing 111 with the motor shaft 118 being longitudinal so that the center of the shaft 118 is parallel to the plane of the earth at the location of a longitudinal chamber 119, preferably made of a transparent material including, but not limited to, plastic.

In this preferred embodiment, the longitudinal chamber 119 is connected to the shaft 118 so that in operation the chamber 119 rotates about its longitudinal axis with the longitudinal axis parallel to the plane of the earth. The chamber 119 is wound with a wire coil 120. The size of the wire coil 120 and number of times it is wound are such that when a square wave current preferably of from 0.1 mA to 1000 mA is supplied to the wire coil 120, a time varying electromagnetic force preferably of from 0.05 gauss to 6 gauss is generated within the chamber 119. The wire coil 120 is connected to a first ring 121 and a second ring 122 at the end of the shaft 118 by wires 123 and 124. These rings 121, 122 are then contacted by a first electromagnetic delivery wire 125 and a second electromagnetic delivery wire 128 in such a manner that the chamber 119 can rotate while the current is constantly supplied to the coil 120. An electromagnetic generating (TVEMF source) device 126 is connected to the wires 125, 128. The electromagnetic generating device 126 supplies a square wave to the wires 125, 128 and coil 120 by adjusting its output by turning an electromagnetic generating device knob 127.

FIG. 3 is a side perspective view of the TVEMF-bioreactor 10 shown in FIG. 2 that may be used in the present invention.

Turning now to the rotating TVEMF-bioreactor 10 illustrated in FIG. 4 with a culture chamber 230 which is preferably transparent and adapted to contain a blood mixture therein, further comprising an outer housing 220 which includes a first 290 and second 291 cylindrically shaped transverse end cap member having facing first 228 and second 229 end surfaces arranged to receive an inner cylindrical tubular glass member 293 and an outer tubular glass member 294. Suitable pressure seals are provided. Between the inner 293 and outer 294 tubular members is an annular wire heater 296 which is utilized for obtaining the proper incubation temperatures for cell growth. The wire heater 296 can also be used as a time varying electromagnetic force source to supply a time varying electric field to the culture chamber 230 or, as depicted in FIG. 5, a separate wire coil 144 can be used to supply a time varying electromagnetic force. The first end cap member 290 and second end cap member 291 have inner curved surfaces adjoining the end surfaces 228, 229 for promoting smoother flow of the mixture within the chamber 230. The first end cap member 290, and second end cap member 291 have a first central fluid transfer journal member 292 and second central fluid transfer journal member 295, respectively, that are rotatably received respectively on an input shaft 223 and an output shaft 225. Each transfer journal member 294, 295 has a flange to seat in a recessed counter bore in an end cap member 290, 291 and is attached by a first lock washer and ring 297, and second lock washer and ring 298 against longitudinal motion relative to a shaft 223, 225. Each journal member 294, 295 has an intermediate annular recess that is connected to longitudinally extending, circumferentially arranged passages. Each annular recess in a journal member 292, 295 is coupled by a first radially disposed passage 278 and second radially disposed passage 279 in an end cap member 290 and 291, respectively, to first input coupling 203 and second input coupling 204. Carrier in a radial passage 278 or 279 flows through a first annular recess and the longitudinal passages in a journal member 294 or 295 to permit access carrier through a journal member 292, 295 to each end of the journal 292, 295 where the access is circumferential about a shaft 223, 225.

Attached to the end cap members 290 and 291 are a first tubular bearing housing 205, and second tubular bearing housing 206 containing ball bearings which relatively support the outer housing 220 on the input 223 and output 225 shafts. The first bearing housing 205 has an attached first sprocket gear 210 for providing a rotative drive for the outer housing 220 in a rotative direction about the input 223 and output 225 shafts and the longitudinal axis 221. The first bearing housing 205, and second bearing housing 206 also have provisions for electrical take out of the wire heater 296 and any other sensor.

The inner filter assembly 235 includes inner 215 and outer 216 tubular members having perforations or apertures along their lengths and have a first 217 and second 218 inner filter assembly end cap member with perforations. The inner tubular member 215 is constructed in two pieces with an interlocking centrally located coupling section and each piece attached to an end cap 217 or 218. The outer tubular member 216 is mounted between the first 217 and second inner filter assembly end caps.

The end cap members 217, 218 are respectively rotatably supported on the input shaft 223 and the output shaft 225. The inner member 215 is rotatively attached to the output shaft 225 by a pin and an intermitting groove 219. A polyester cloth 224 with a ten-micron weave is disposed over the outer surface of the outer member 216 and attached to O-rings at either end. Because the inner member 215 is attached by a coupling pin to a slot in the output drive shaft 225, the output drive shaft 225 can rotate the inner member 215. The inner member 215 is coupled by the first 217 and second 218 end caps that support the outer member 216. The output shaft 225 is extended through bearings in a first stationary housing 240 and is coupled to a first sprocket gear 241. As illustrated, the output shaft 225 has a tubular bore 222 that extends from a first port or passageway 289 in the first stationary housing 240 located between seals to the inner member 215 so that a flow of fluid carrier can be exited from the inner member 215 through the stationary housing 240.

Between the first 217 and second 218 end caps for the inner member 235 and the journals 292, 295 in the outer housing 220, are a first 227 and second 226 hub for the blade members 50a and 50b. The second hub 226 on the input shaft 223 is coupled to the input shaft 223 by a pin 231 so that the second hub 226 rotates with the input shaft 223. Each hub 227, 226 has axially extending passageways for the transmittal of carrier through a hub.

The input shaft 223 extends through bearings in the second stationary housing 260 for rotatable support of the input shaft 223. A second longitudinal passageway 267 extends through the input shaft 223 to a location intermediate of retaining washers and rings that are disposed in a second annular recess 232 between the faceplate and the housing 260. A third radial passageway 272 in the second end cap member 291 permits fluid carrier in the recess to exit from the second end cap member 291. While not shown, the third passageway 272 connects through piping and a Y joint to each of the passages 278 and 279.

A sample port is shown in FIG. 4, where a first bore 237 extending along a first axis intersects a corner 233 of the chamber 230 and forms a restricted opening 234. The bore 237 has a counter bore and a threaded ring at one end to threadedly receive a cylindrical valve member 236. The valve member 236 has a complimentarily formed tip to engage the opening 234 and protrude slightly into the interior of the chamber 230. An O-ring 243 on the valve member 236 provides a seal. A second bore 244 along a second axis intersects the first bore 237 at a location between the O-ring 243 and the opening 234. An elastomer or plastic stopper 245 closes the second bore 244 and can be entered with a hypodermic syringe for removing a sample. To remove a sample, the valve member 236 is backed off to access the opening 234 and the bore 244. A syringe can then be used to extract a sample and the opening 234 can be reclosed. No outside contamination reaches the interior of the TVEMF-bioreactor 10.

In operation, carrier is input to the second port or passageway 266 to the shaft passageway and thence to the first radially disposed 278 and second radially disposed passageways 279 via the third radial passageway 272. When the carrier enters the chamber 230 via the longitudinal passages in the journals 292, 294 the carrier impinges on an end surface 228, 229 of the hubs 227, 226 and is dispersed radially as well as axially through the passageways in the hubs 227, 226. Carrier passing through the hubs 227, 226 impinges on the end cap members 217, 218 and is dispersed radially. The flow of entry fluid carrier is thus radially outward away from the longitudinal axis 221 and flows in a toroidal fashion from each end to exit through the polyester cloth 224 and openings in filter assembly 235 to exit via the passageways 266 and 289. By controlling the rotational speed and direction of rotation of the outer housing 220, chamber 230, and inner filter assembly 235 any desired type of carrier action can be obtained. Of major importance, however, is the fact that a clinostat operation can be obtained together with a continuous supply of fresh fluid carrier.

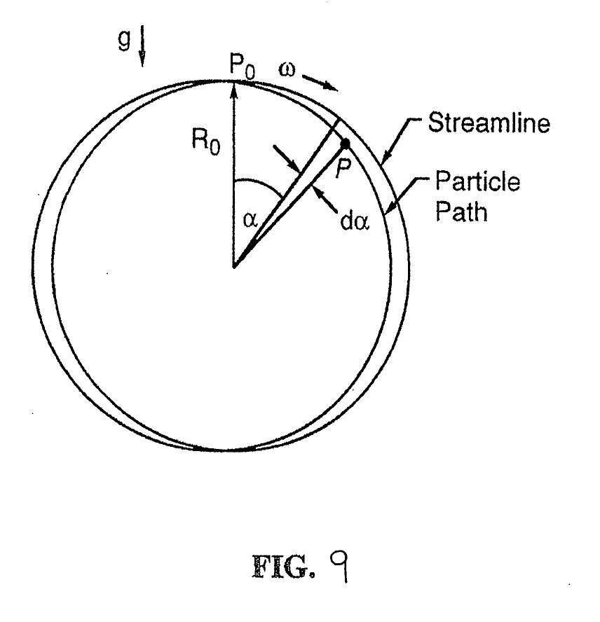

If a time varying electromagnetic force is not applied using the integral annular wire heater 296, it can be applied by another preferred time varying electromagnetic force source. For instance, FIGS. 6-8 illustrate a time varying electromagnetic force source 140 which provides an electromagnetic force to a cell culture in a bioreactor which does not have an integral time varying electromagnetic force, but rather has an adjacent time varying electromagnetic force source. Specifically, FIG. 6 is a preferred embodiment of a time varying electromagnetic force source 140. FIG. 6 is an elevated side perspective of the time varying electromagnetic force source 140 which comprises a support base 145, a cylinder coil support 146 supported on the base 145 with a wire coil 147 wrapped around the support 146. FIG. 7 is a front perspective of the time varying electromagnetic force source 140 illustrated in FIG. 6. FIG. 8 is a front perspective of the time varying electromagnetic force source 140, which illustrates that in operation, an entire bioreactor 148 is inserted into a cylinder coil support 146 which is supported by a support base 145 and which is wound by a wire coil 147. It is not necessary that the TVEMF source comprise a coil, but may also preferably comprise at least one loop, each of which emit a TVEMF signal. Since the time varying electromagnetic force source 140 is adjacent to the bioreactor 148, the time varying electromagnetic force source 140 can be reused. In addition, since the time varying electromagnetic force source 140 is adjacent to the bioreactor 148, the source 140 can be used to generate an electromagnetic force in all types of bioreactors, preferably rotating.

Furthermore, in operation a preferred embodiment of the present invention contemplates that an electromagnetic generating source is turned on and adjusted so that the output generates the desired electromagnetic field in the blood mixture-containing chamber. One embodiment of the TVEMF source is that it can be configured to emit a TVEMF signal exhibiting a relatively high magnetic field amplitude (between about 10 to 100 Gauss) and exhibiting a magnetic slew rate greater than 1000 Gauss per second. Another embodiment of the TVEMF source is that it can be configured to emit a TVEMF signal exhibiting a relatively low magnetic field amplitude (between about 0.1 to 10 Gauss) along a bipolar square wave function at a frequency of between 1 to 100 Hz. Yet another embodiment of the TVEMF source is that it can also be configured to emit a TVEMF signal exhibiting relatively low magnetic field amplitude (between about 0.1 to 10 Gauss) along a square wave function having a duty cycle between about 0.1 to 99.9 percent. Still another embodiment of the TVEMF source is that it can also be configured to emit a TVEMF signal exhibiting a magnetic field having a magnetic slew rate greater than about 1000 Gauss per second that has a active duty pulse duration of less than 1 ms. Still yet another embodiment of the TVEMF source is that it can also be configured to emit a TVEMF signal exhibiting a magnetic field having a magnetic slew rate greater than about 50 Gauss per second exhibiting a bipolar pulses having an active duty cycle of less than 1%. Even still yet another embodiment of the TVEMF source is that it can also be configured to emit a TVEMF signal exhibiting a magnetic field between about 1 to 100 Gauss peak-to-peak and having a magnetic slew rate bipolar pulses with an active duty cycle of less than 1%. Still another embodiment of the TVEMF source, for instance comprising a solenoid coil, is that it can also be configured to emit a TVEMF signal exhibiting a time-dependent magnetic field exhibiting a relatively uniform (not varying by more than 5%) magnetic field strength throughout the cell mixture contents.

However, these parameters are not meant to be limiting to the TVEMF of the present invention, and as such may vary based on other aspects of this invention. TVEMF may be measured for instance by standard equipment such as an EN131 Cell Sensor Gauss Meter.

As various changes could be made in rotating bioreactors subjected to a time varying electromagnetic force as are contemplated in the present invention, without departing from the scope of the invention, it is intended that all matter contained in the above description be interpreted as illustrative and not limiting.

### DETAILED DESCRIPTION OF THE PREFERRED EMBODIMENTS OF THE INVENTION

The present invention is related to a method of expanding peripheral blood stem cells without substantial differentiation. This invention may be more fully described by the preferred embodiment as hereinafter described, but is not intended to be limited thereto. Peripheral blood cells are removed from a patient. A subpopulation of these cells is currently referred to as adult stem cells. The peripheral blood cells, or any subset thereof, are placed in a bioreactor as described herein. The bioreactor vessel is rotated 360° about a substantially horizontal longitudinal central axis at a speed that provides for suspension of the peripheral blood cells to maintain each cell in a discrete microenvironment essentially without any turbulence, and with low shear stress. During the time that the cells are in the reactor, they may be fed nutrients, exposed to hormones, cytokines, or growth factors, and/or genetically modified, and toxic materials are preferably removed. The toxic materials typically removed are from peripheral blood cells comprising the toxic granular material of dying cells and the toxic material of granulocytes and macrophages. In addition to providing a rotating bioreactor for the suspension and expansion of cells, the present invention also contemplates the addition of TVEMF to the rotating bioreactor.

The following definitions are meant to aid in the description and understanding of the defined terms in the context of the present invention. The definitions are not meant to limit these terms to less than is described throughout this application. Furthermore, several definitions are included relating to TVEMF—all of the definitions in this regard should be considered to complement each other, and not construed against each other.

As used throughout this application, the term “rotating bioreactor” refers to a bioreactor that can be rotated about a substantially horizontal axis, horizontal to the plane of the earth, and about the culture chambers longitudinal axis. In addition, the rotating bioreactor is rotated 360 degrees in one direction so that the cells contained therein are suspended in discrete microenvironments with very little, if any, turbulence and low shear stress. A short recess is permitted wherein culture media can be refreshed, samples taken, or for other reasons, without disturbing the suspension of the cells in the rotating bioreactor. The bioreactors of the present invention, with and without TVEMF, provide a three-dimensional environment wherein the entire volume of the culture chamber is filled so as to provide essentially zero headspace. In addition, the rotating bioreactor essentially mimics a microgravity situation. A rotating bioreactor can be made a rotating TVEMF bioreactor with the addition of TVEMF.

As used throughout this application, the term “adult stem cell” refers to a pluripotent, totipotent, and/or multipotent cell that is undifferentiated and that may give rise to more undifferentiated cells and is also capable to giving rise to differentiated cells, but only if directed to. With regard to the present invention, an adult stem cell is preferably a CD133+ cell, more preferably a CD34+, and most preferably a non-terminally differentiated peripheral blood stem cell.

As used throughout this application, the term “blood” refers to peripheral blood. “Peripheral blood” is systemic blood; that is, blood that circulates, or has circulated, systemically in a mammal. The mammal is not meant to be a fetus. For the purposes of the present invention, there is no reason to distinguish between peripheral blood located at different parts of the same circulatory loop.

As used throughout this application, the term “peripheral blood cell” refers to a cell from blood. Blood cells capable of replication may undergo expansion, preferably TVEMF-expansion in a TVEMF-bioreactor, and may be present in compositions of the present invention.

As used throughout this application, the term “peripheral blood stem cell” refers to an adult stem cell from peripheral blood. Peripheral blood stem cells are adult stem cells, which as mentioned above are also known as somatic stem cells, and are not embryonic stem cells derived directly from an embryo. Preferably, a peripheral blood stem cell of the present invention is a CD34+ cell, more preferably a CD133+ cells, and most preferably a non-terminally differentiated cell.

As used throughout this application, the term “peripheral blood stem cell composition”, or reference thereto, refers to peripheral blood stem cells and a carrier of some sort, whether a pharmaceutically acceptable carrier, plasma, peripheral blood, albumin, cell culture medium, growth factor, copper chelating agent, hormone, buffer, cryopreservative, or some other substance. Reference to naturally-occurring peripheral blood is preferably to compare peripheral blood stem cells of the present invention with their original peripheral blood source. However, if such a comparison is not available, then naturally-occurring peripheral blood may refer to average or typical characteristics of such peripheral blood, preferably of the same mammalian species as the source of the peripheral blood stem cells of this invention.

A “pharmaceutical peripheral blood stem cell composition” of this invention is a peripheral blood stem cell composition that is suitable for administration into a mammal, preferably into a human. Such a composition has a therapeutically effective amount of expanded (preferably TVEMF-expanded) peripheral blood stem cells. A therapeutically effective amount of expanded peripheral blood stem cells is (also discussed elsewhere herein) preferably at least 1000 stem cells, more preferably at least 10⁴ stem cells, even more preferably at least 10⁵ stem cells, and even more preferably in an amount of at least 10⁷ to 10⁹ stem cells, or even more stem cells such as 10¹² stem cells. Administration of such numbers of expanded stem cells may be in one or more doses. As indicated throughout this application, the number of stem cells administered to a patient may be limited to the number of stem cells originally available in source peripheral blood, as multiplied by expansion according to this invention. Without being bound by theory, it is believed that stem cells not used by the body after administration will simply be removed by natural body systems. It should also be noted that another preferred embodiment provides for the culturing of cells wherein the cells are expanded for a time without regard to the number of cells in the culture, but instead, where the expanded cells are cultured and thereby have unique characteristics that are suitable to the repair of mammalian tissue and/or function.

As used throughout this application, the term “peripheral blood mixture” refers to a mixture of peripheral blood/peripheral blood cells with a substance that helps the cells to expand, such as a medium for growth of cells, that may be placed in a bioreactor (for instance in a cell culture chamber). The peripheral blood cells may be present in the peripheral blood mixture simply by mixing whole peripheral blood with a substance such as a cell culture medium. Also, the peripheral blood mixture may be made with a cellular preparation from peripheral blood, as described throughout this application, such as a “buffy coat,” containing peripheral blood stem cells. Preferably, the peripheral blood mixture comprises peripheral blood stem cells and Dulbecco's medium (DMEM). Preferably, at least half of the peripheral blood mixture is a cell culture medium such as DMEM.

As used throughout this application, the term “TVEMF” refers to “Time Varying Electromagnetic Force”.

As used throughout this application, the term “TVEMF-bioreactor” refers to a rotating bioreactor to which TVEMF is applied, as described more fully in the Description of the Drawings, above. The TVEMF applied to a bioreactor is preferably as disclosed herein. See for instance FIGS. 2, 3, 4 and 5 herein for examples (not meant to be limiting) of a TVEMF-bioreactor. In a simple embodiment, a TVEMF-bioreactor of the present invention provides for the rotation of an enclosed peripheral blood mixture at an appropriate TVEMF and allows the peripheral blood cells (including stem cells) therein to expand. Preferably, a TVEMF-bioreactor allows for the exchange of growth medium (preferably with additives) and for oxygenation of the peripheral blood mixture. The TVEMF-bioreactor provides a mechanism for expanding cells for several days or more. The TVEMF-bioreactor subjects cells in the bioreactor to TVEMF, so that TVEMF is passed through or otherwise exposed to the cells, the cells thus undergoing TVEMF-expansion. The rotation of the TVEMF-bioreactor during TVEMF-expansion is preferably at a rate of 5 to 120 rpm, more preferably 10 to 30 rpm, to foster minimal wall collision frequency and intensity so as to maintain the bloodstream cell three-dimensional geometry and cell-to-cell support and cell-to-cell geometry.

As used throughout this application, the term “expanded peripheral blood cells” refers to peripheral blood cells increased in number (ie concentration) and/or cultured after being placed in a rotating bioreactor. “TVEMF expanded peripheral blood cells” refers to peripheral blood cells TVEMF-expanded in a TVEMF bioreactor wherein the cells are increased in number and/or cultured in the rotating TVEMF-bioreactor and subjected to a TVEMF. The increase in number of cells is the result of cell replication in the bioreactor, so that the total number of cells in the bioreactor increases. The increase in number of cells is expressly not due to a simple reduction in volume of fluid, for instance, reducing the volume of peripheral blood from 70 ml to 10 ml and thereby increasing the number of cells per ml. By increasing in number it is intended that the cells replicate (and thereby grow in number). Substantially all peripheral blood stem cells (preferably CD34+, more preferably CD133+, and most preferably non-terminally differentiated stem cells) preferably expand without undergoing further differentiation. “Substantially all” is meant to refer to at least 70%, preferably at least 80%, more preferably at least 90%, even more preferably at least 95%, even more preferably at least 97%, and most preferably at least 99% of the stem cells do not differentiate.

In a preferred embodiment, the number of cells expanded not important. In such an embodiment, it is contemplated that by culturing the cells in the rotating bioreactor (with or without TVEMF), the cells will have enhanced repairing and regenerating capabilities. For instance, the cells may have enhanced tissue repairing characteristics or tissue function repairing characteristics by being cultured in the rotating bioreactor. If the preference is to culture the cells then a user may not focus on the number of cells expanded. For instance, if the culture in the rotating bioreactor is the focus of the method, then zero additional cells, less than the number that were placed in the rotating bioreactor, and at least one more than number that were placed in the bioreactor may all be acceptable numbers.

As used throughout this application, the term “expansion” refers to the process of increasing the number of peripheral blood cells in a bioreactor and/or culturing the peripheral blood cells, preferably peripheral blood stem cells, in a rotating bioreactor, preferably a TVEMF-bioreactor whereing the cells are subjected to a TVEMF. Preferably, the increase in number of peripheral blood stem cells is at least 7 times the number of peripheral blood stem cells that were placed into the rotating bioreactor, preferably TVEMF, for expansion. The expansion of peripheral blood stem cells in a rotating bioreactor according to the present invention provides for peripheral blood stem cells that maintain, or have essentially the same, three-dimensional geometry and cell-to-cell support and cell-to-cell geometry as peripheral blood stem cells prior to expansion, and also have a unique phenotypic expression due to the three-dimensional culture.

Other aspects of expansion may also provide the exceptional characteristics of the peripheral blood stem cells of the present invention, which is why the number or expanded cells may not be the focus of the expansion process, but rather, the three-dimensional culture environment in the rotating system. Not to be bound by theory, in one embodiment, expansion provides for high concentrations of peripheral blood stem cells that maintain their three-dimensional geometry and cell-to-cell support while at the same time adopt a unique phenotypic expression as a result of the culture environment in which they are expanded. TVEMF may affect some properties of stem cells during TVEMF-expansion, for instance up-regulation of genes promoting growth, or down regulation of genes preventing growth. Overall, expansion results in promoting growth in number and/or culture but not differentiation overall. It is also contemplated that before expansion, the cells may preferably be cultured in a two-dimensional or preferably in a three-dimensional system for a preferred amount of time before placing the cells in the rotating system for expansion.

Some genes that are up regulated may preferably include, but are not limited to, those coding for membrane proteins such as proteoglycan 3, CYPiB, IL9R, HBA1, and RHAG; coding for cytoskeletal proteins such as SPTA1, ANK1; enzymes such as NCALD, LSS, PDE4B, SPUB, ELA2, HGD, ADAMDEC1, HMGCS1, COVA1, and PFKB4; nuclear/transcription factors such as Pirin; and others such as S100A8, A9. Some genes that are down regulated may preferably include, but are not limited to, membrane proteins such as IL2R, IL17 R, EV127, TGFR3, FCGR1A, MRC1, CCR1, CRL4, FER1L3, EMP1, and THBD; transport proteins such as ABC1A and ABCG1; glycoproteins/cell surface proteins such as Versican, CD1c, CD14, areg, z391G, hm12, and CLECSF5; cytoskeletal transduction proteins such as SKG1; secreted proteins such as SCYA3, gro3, and galectin3; nuclear transcription factors such as KRML, LOC51713, KLF4, and EGR1; and HMOX1 and BPHL. Preferably, the up regulated genes are up regulated up to 2 fold, and preferably the down regulated genes are down regulated up to four fold.

As used throughout this application, the term “TVEMF-expanded cell” refers to a cell that has been subjected to the process of TVEMF-expansion. A TVEMF-expanded cell retains some core properties of the same cell in vivo, but also has a unique phenotypic expression as a result of the TVEMF-expansion process including suspension in the rotating TVEMF-bioreactor. As afore mentioned, in a preferred embodiment, the expanded cell may preferably be cultured rather than expanded in number. In another preferred embodiment, the cells are expanded without the addition of TVEMF.

Throughout this application, the terms “repair”, “replenish” and “regenerate” are used. These terms are not meant to be mutually exclusive, but rather related to overall tissue repair.

Throughout this application, reference to the repair of tissue, treatment of disease or condition, are not meant to be exclusive but rather relate to the objective of overall tissue repair where improvement in tissue results from administration of stem cells as discussed herein. While the present invention is directed in part to diseases or conditions that are symptomatic, and possibly life-threatening, the present invention is also meant to include treatment of minor repair, and even prevention/prophylaxis of disease/condition by early introduction of expanded stem cells, before symptoms or problems in the mammal's (preferably human's) health are notice.

As used throughout this application, the term “toxic substance” or related terms may refer to substances that are toxic to a cell, preferably a peripheral blood stem cell; or toxic to a patient. In particular, the term toxic substance refers to dead cells, macrophages, as well as substances that may be unique or unusual in peripheral blood (for instance, sickle cells in peripheral blood, or other tissue or waste). Other toxic substances are discussed throughout this application. Removal of these substances from peripheral blood is well-known in the art.

As used throughout this application, the term “apheresis of bone marrow” refers to inserting a needle into bone and extracting bone marrow. Such apheresis is well-known in the art.

As used throughout this application, the term “autologous” refers to a situation in which the donor (source of peripheral blood stem cells prior to expansion) and recipient are the same mammal. The present invention includes autologous tissue repair and replenishment.

As used throughout this application, the term “allogeneic” refers to a situation in which the donor (source of peripheral blood stem cells prior to expansion) and recipient are not the same mammal. The present invention includes allogeneic tissue repair and replenishment.

As used throughout this application, the term “cell-to-cell geometry” refers to the geometry of cells including the spacing, distance between, and physical relationship of the cells relative to one another. For instance, expanded stem cells of this invention stay in relation to each other as in the body. The expanded cells are within the bounds of natural spacing between cells, in contrast to for instance two-dimensional expansion containers, where such spacing is not kept.

As used throughout this application, the term “cell-to-cell support” refers to the support one cell provides to an adjacent cell. For instance, healthy tissue and cells maintain interactions such as chemical, hormonal, neural (where applicable/appropriate) with other cells in the body. In the present invention, these interactions are maintained within normal functioning parameters, meaning they do not for instance begin to send toxic or damaging signals to other cells (unless such would be done in the natural peripheral blood environment).

As used throughout this application, the term “three-dimensional geometry” refers to the geometry of cells in a three-dimensional state (same as or very similar to their natural state), as opposed to two-dimensional geometry for instance as found in cells grown in a Petri dish, where the cells become flattened and/or stretched.

For each of the above three definitions, relating to maintenance of cell-to-cell support and geometry and three dimensional geometry of stem cells of the present invention, the term “essentially the same” means that normal geometry and support are provided in expanded cells of this invention, so that the cells are not for instance changed in such a way as to be disfunctional, unable to repair tissue or toxic or harmful to other cells.

Other statements referring to the above-defined terms or other terms used throughout this application are not meant to be limited by the above definitions, and may contribute to the definitions. Information relating to various aspects of this invention is provided throughout this application, and is not meant to be limited only to the section to which it is contained, but is meant to contribute to an understanding of the invention as a whole.

This invention may be more fully described by the preferred embodiment(s) as hereinafter described, but is not intended to be limited thereto.

### Operative Method

### Preparing a Peripheral Blood Mixture

Blood is collected from a mammal, preferably a primate mammal, and more preferably a human, for instance as described throughout this application, and preferably according to the syringe method as well known in the art. Peripheral blood may be collected, for instance, and expanded immediately and used, or cryopreserved in expanded or unexpanded form for later use. Peripheral blood would only be removed from a human in an amount that would not be threatening to the subject. Preferably, about 10 to about 500 ml peripheral blood is collected; more preferably, 100-300 ml, even more preferably, 150-200 ml. The collection of peripheral blood according to this invention is not meant to be limiting, but can also include for instance other means of directly collecting mammalian peripheral blood, pooling peripheral blood from one or more sources, indirectly collecting blood for instance by acquiring the blood from a commercial or other source, including for instance cryopreserved peripheral blood from a “blood bank”, or blood otherwise stored for later use.

Typically, when directly collected from a mammal, peripheral blood is drawn into one or more syringes, preferably containing anticoagulants. The blood may be stored in the syringe or transferred to another vessel. Blood may then be separated into its parts; white blood cells, red blood cells, and plasma. This is either done in a centrifuge (an apparatus that spins the container of blood until the blood is divided) or by sedimentation (the process of injecting sediment into the container of blood causing the blood to separate). Second, once the blood is divided with the red blood cells (RBC) on the bottom, white blood cells (WBC) in the middle, and the plasma on top, the white blood cells are removed for storage. The middle layer, also known as the “buffy coat” contains the blood stem cells of interest; the other parts of the blood are not needed. For some blood banks, this will be the extent of their processing. However, other banks will go on to process the buffy coat by removing the mononuclear cells (in this case, a subset of white blood cells) from the WBC. While not everyone agrees with this method, there is less to store and less cryogenic nitrogen is needed to store the cells.

Another method for separating blood cells is to subject all of the collected peripheral blood to one or more (preferably three) rounds of continuous flow leukapheresis in a separator such as a Cobe Spectra cell separator. Such processing will separate blood cells having one nucleus from other blood cells. The stem cells are part of the group having one nucleus. Other methods for the separation of blood cells are known in the art.

It is preferable to remove the RBC from the peripheral blood sample. While people may have the same HLA type (which is needed for the transplanting of stem cells), they may not have the same blood type. By removing the RBC, adverse reactions to a stem cell transplant can be minimized. By eliminating the RBC, therefore, the stem cell sample has a better chance of being compatible with more people. RBC can also burst when they are thawed, releasing free hemoglobin. This type of hemoglobin can seriously affect the kidneys of people receiving a transplant. Additionally, the viability of the stem cells are reduced when RBC rupture.

Also, particularly if storing blood cryogenically or transferring the blood to another mammal, the peripheral blood may be tested to ensure no infectious or genetic diseases, such as HIV/AIDS, hepatitis, leukemia or immune disorder, is present. If such a disease exists, the blood may be discarded or used with associated risks noted for a future user to consider.

In still another embodiment of this invention, peripheral blood cells may be obtained from a donor. Prior to collection, the donor is treated with G-CSF (preferably in an amount of 0.3 ng to 5 ug, more preferably 1 ng/kg to 100 ng/kg, even more preferably 5 ng/kg to 20 ng/kg, and even more preferably 6 ng/kg) every 12 hr over 3 days and then once on day 4. In a preferred method, a like amount of GM-CSF is also administered. Other alternatives are to use GM-CSF alone, or other growth factor molecules, interleukins. Blood is then collected from the donor, and may be used whole in a peripheral blood mixture or first separated into cellular parts as discussed throughout this application, where the cellular part including stem cells is used to prepare the peripheral blood mixture to be expanded. Cells may be separated, for instance, by subjecting the donor's total blood volume to 3 rounds of continuous-flow leukapheresis through a separator, such as a Cobe Spectra cell separator. Preferably, the expanded stem cells are reintroduced into the same donor, where the donor is in need of tissue repair as discussed herein. However, allogeneic introduction may also be used, as also indicated herein. Other pre-collection administrations will also be evident to those skilled in the art.

Preferably, red blood cells are removed from the peripheral blood and the remaining cells including peripheral blood stem cells are placed with an appropriate media in a bioreactor (see “peripheral blood mixture”) such as that described herein. In a more preferred embodiment of this invention, only the “buffy coat” (which includes peripheral blood stem cells, as discussed throughout this application) described above is the cellular material placed in the bioreactor. Other embodiments include removing other non-stem cells and components of the peripheral blood, to prepare different peripheral blood preparation(s). Such a peripheral blood preparation may even have, as the only remaining peripheral blood component, peripheral blood stem cells. Removal of non-stem cell types of peripheral blood cells may be achieved through negative separation techniques, such as but not limited to sedimentation and centrifugation. Many negative separation methods are well-known in the art. However, positive selection techniques may also be used, and are preferred in this invention. Methods for removing various components of the peripheral blood and positively selecting for, but not limited to, CD34+ and/or other markers such as CD133+, are known in the art, and may be used so long as they do not lyse or otherwise irreversibly harm the desired peripheral blood stem cells. For instance, an affinity method selective for CD34+ may be used. Preferably, a “buffy coat” as described above is prepared from peripheral blood, and the peripheral blood stem cells therein separated from the buffy coat for expansion.

The collected peripheral blood, or desired cellular parts as discussed above, must be placed into a rotating bioreactor for expansion, or preferably a TVEMF-bioreactor for TVEMF-expansion, to occur. As discussed above, the term “peripheral blood mixture” comprises a mixture of peripheral blood (or desired cellular part, for instance peripheral blood without red blood cells) with a substance that allows the cells to expand, such as a medium for growth of cells that will be placed in a bioreactor. Cell culture media, media that allow cells to grow and expand, are well-known in the art. Preferably, the substance that allows the cells to expand is cell culture media, more preferably Dulbecco's medium. The components of the cell media must, of course, not kill or damage the stem cells. Other components may also be added to the peripheral blood mixture prior to or during expansion. For instance, the peripheral blood may be placed in the bioreactor with Dulbecco's medium and further supplemented with 5% (or some other desired amount, for instance in the range of about 1% to about 10%) of human serum albumin. Other additives to the peripheral blood mixture, including but not limited to growth factor, copper chelating agent, cytokine, hormone and other substances that may enhance expansion may also be added to the peripheral blood outside or inside the bioreactor before being placed in the bioreactor.

Preferably, the entire volume of a peripheral blood collection from one individual (preferably human peripheral blood in an amount of about 10 ml to about 500 ml, more preferably about 100 ml to about 300 ml, even more preferably about 150 to about 200 ml peripheral blood) is mixed with a cell culture medium such as Dulbecco's medium (DMEM) and supplemented with 5% human serum albumin to prepare a peripheral blood mixture for expansion. For instance, for a 50 to 100 ml peripheral blood sample, preferably about 25 to about 100 ml DMEM/5% human serum albumin is used, so that the total volume of the peripheral blood mixture is about 75 to about 200 ml when placed in the bioreactor. As a general rule, the more peripheral blood that may be collected, the better; if a collection from one individual results in more than 100 ml, the use of all of that peripheral blood is preferred. Where a larger volume is available, for instance by pooling peripheral blood (from the same or different source), more than one dose may be preferred. The use of a perfusion bioreactor is particularly useful when peripheral blood collections are pooled and expanded together.

A copper chelating agent of the present invention may be any non-toxic copper chelating agent, and is preferably Penicillamine or Trientine Hydrochloride. More preferably, the Penicillamine is D(−)-2-Amino-3-Mercaptor-3-Methylbutanic Acid (Sigma-Aldrich), dissolved in DMSO and added to the peripheral blood mixture in an amount of about 10 ppm. The copper chelating agent may also be administered to a mammal, where peripheral blood will then be directly collected from the mammal. Preferably such administration is more than one day, more preferably more than two days, before collecting peripheral blood from the mammal. The purpose of the copper chelating agent, whether added to the peripheral blood mixture itself or administered to a peripheral blood donor mammal, or both, is to reduce the amount of copper in the peripheral blood prior to expansion. Not to be bound by theory, it is believed that the decrease in amount of available copper may enhance expansion, including TVEMF-expansion.

The term “placed in a bioreactor” is not meant to be limiting and also applies to peripheral blood “placed in a TVEMF-bioreactor”—the peripheral blood mixture may be made entirely outside of the bioreactor and then the mixture placed inside the bioreactor. Also, the peripheral blood mixture may be entirely mixed inside the bioreactor. For instance, the peripheral blood (or a cellular portion thereof) may be placed in the bioreactor and supplemented with Dulbecco's medium and 5% human serum albumin either already in the bioreactor, added simultaneously to the bioreactor, or added after the peripheral blood to the bioreactor.

A preferred peripheral blood mixture of the present invention comprises the following: peripheral blood stem cells isolated from the buffy coat of a peripheral blood sample and Dulbecco's medium which, with the cells, is about 150-250 ml, preferably about 200 ml total volume. Even more preferably, G-CSF (Granulocyte-Colony Stimulating Factor) is included in the peripheral blood mixture. Preferably, G-CSF is present in an amount sufficient to enhance expansion of peripheral blood stem cells. Even more preferably, the amount of G-CSF present in the peripheral blood mixture prior to TVEMF-expansion is about 25 to about 200 ng/ml peripheral blood mixture, more preferably about 50 to about 150 ng/ml, and even more preferably about 100 ng/ml.

### Operative Method

### Expansion

In use, the rotation of a bioreactor (TVEMF or otherwise) provides a stabilized culture environment into which cells may be introduced, suspended, maintained, and expanded with improved retention of delicate three-dimensional structural integrity by simultaneously minimizing the fluid shear stress, providing three-dimensional freedom for cell and substrate spatial orientation, and increasing localization of cells in a particular spatial region for the duration of the expansion (hereinafter referred to as “three criteria”). The rotating TVEMF-bioreactor also provides these three criteria, and at the same time, exposes the cells to a TVEMF. Of particular interest to the present invention is the dimension of the culture chamber, the sedimentation rate of the cells, the rotation rate, the external gravitational field, and the TVEMF.

The stabilized culture environment referred to in the operation of present invention is that condition in the culture medium, particularly the fluid velocity gradients, prior to introduction of cells, which will support a nearly uniform suspension of cells upon their introduction thereby creating a three-dimensional culture upon addition of the cells. In a preferred embodiment, the culture medium is initially stabilized into a near solid body horizontal rotation 360 degrees about an axis within the confines of a similarly rotating chamber wall of a rotatable bioreactor. The rotating continues in the same direction about the axis. The chamber walls are set in motion relative to the culture medium so as to initially introduce essentially no fluid stress shear field therein. Cells are introduced to, and move through, the culture medium in the stabilized culture environment thus creating a three-dimensional culture. The cells move under the influence of gravity, centrifugal, and coriolus forces, and the presence of cells within the culture medium of the three-dimensional culture induces secondary effects to the culture medium. The motion of the culture medium with respect to the culture chamber, fluid shear stress, and other fluid motions, is due to the presence of these cells within the culture medium.

In most cases the cells with which the stabilized culture environment is primed sediment at a slow rate preferably under 0.1 centimeter per second. It is therefore possible, at this early stage of the three-dimensional culture, to select from a broad range of rotational rates (preferably of from about 2 to about 30 RPM) and chamber diameters (preferably of from about 0.5 to about 36 inches). Preferably, the slowest rotational rate is advantageous because it minimizes equipment wear and other logistics associated with handling the three-dimensional culture. The preferred speed of the present invention is of from 5 to 120 RPM, and more preferably from 10 to 30 RPM.

Not to be bound by theory, rotation about a substantially horizontal axis with respect to the external gravity vector at an angular rate optimizes the orbital path of cells suspended within the three-dimensional culture. The progress of the three-dimensional culture is preferably assessed by a visual, manual, or automatic determination. An increase in the density of cells may require appropriate adjustment of the rotation speed in order to optimize the particular paths. An increase in density is related to an increase in the number of cells in the culture chamber. The rotation of the culture chamber optimally controls collision frequencies, collision intensities, and localization of the cells in relation to other cells and also the limiting boundaries of the culture chamber of the rotatable bioreactor. In order to control the rotation, if the cells are observed to excessively distort inwards on the downward side and outwards on the upwards side then the revolutions per minute (“RPM”) may preferably be increased. If the cells are observed to centrifugate excessively to the outer walls then the RPM may preferably be reduced. Optimally, the zero-head space of the three-dimensional culture provides a space wherein cells may preferably be distributed throughout the volume of culture medium effectively utilizing the full culture chamber capacity.

The cell sedimentation rate and the external gravitations field place a lower limit on the fluid shear stress obtainable, even within the operating range of the present invention, due to gravitationally induced drift of the cells through the culture medium of the three-dimensional culture. Calculations and measurements place this minimum fluid shear stress very nearly to that resulting from the cells' terminal sedimentation velocity (through the culture medium) for the external gravity field strength. Centrifugal and coriolis induced motion [classical angular kinematics provide the following equation relating the Coriolis force to an object's mass (m), its velocity in a rotating frame (vᵣ) and the angular velocity of the rotating frame of reference (□): F_{Coliolis}=−2 m (w×vᵣ)] along with secondary effects due to cell and culture medium interactions, act to further degrade the fluid shear stress level as the cells expand.

Not to be bound by theory, but an environment that is substantially similar to microgravity may be obtained in the rotating bioreactor. In order to obtain the minimal fluid shear stress level it is preferable that the culture chamber be rotated at substantially the same rate as the culture medium. Not to be bound by theory, but this minimizes the fluid velocity gradient induced upon the three-dimensional culture. It is advantageous to control the rate of expansion in order to maintain the cell density (and associated sedimentation rate) within a range for which the rate of expansion is able to satisfy the three criteria. In addition, transient disruptions of the expansion process are permitted and tolerated for, among other reasons, logistical purposes during initial system priming, sample acquisition, system maintenance, and culture termination.

Rotating cells about an axis substantially perpendicular to gravity can produce a variety of sedimentation rates, all of which according to the present invention remain spatially localized in distinct regions for extended periods of time ranging from seconds (when sedimentation characteristics are large) to hours or days (when sedimentation differences are small). Not to be bound by theory, but this allows these cells sufficient time to interact and associate as necessary with each other in a three-dimensional culture. Preferably, cells undergo expansion for at least 4 days, more preferably from about 7 days to about 14 days, most preferably from about 7 days to about 10 days, even more preferably about 7 days. Expansion may continue in a bioreactor (TVEMF or otherwise) for up to 160 days. While expansion may occur for even longer than 160 days, such a lengthy expansion is not a preferred embodiment of the present invention. Preferably, expansion may continue in a rotatable bioreactor to produce a number of cells that is at least 7 times the original number of cells that were placed in the rotatable bioreactor.

Culture chamber dimensions also influence the path of cells in the three-dimensional culture of the present invention. A culture chamber diameter is preferably chosen which has the appropriate volume, preferably of from about 15 ml to about 2 L for the intended three-dimensional culture and which will allow a sufficient seeding density of cells. Not to be bound by theory, but the outward cells drift due to centrifugal force is exaggerated at higher culture chamber radii and for rapidly sedimenting cells.

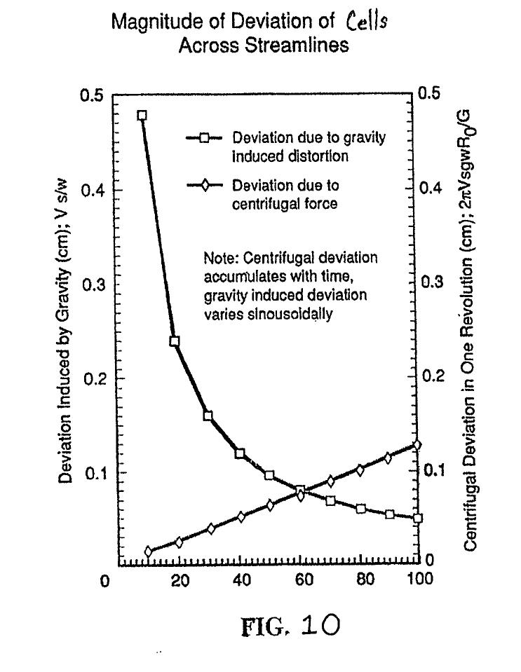

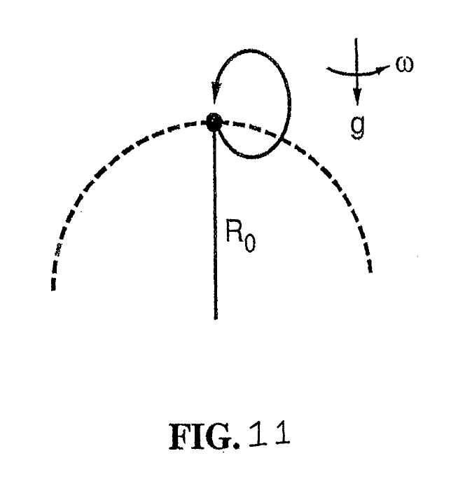

The path of the cells in the three-dimensional culture has been analytically calculated incorporating the cell motion resulting from gravity, centrifugation, and coriolus effects. A computer simulation of these governing equations allows the operator to model the process and select parameters acceptable (or optimal) for the particular planned three-dimensional culture. FIG. 9 shows the typical shape of the cell orbit as observed from the external (non-rotating) reference frame. FIG. 10 is a graph of the radial deviation of a cell from the ideal circular streamline plotted as a function of RPM (for a typical cell sedimenting at 0.5 cm per second terminal velocity). This graph (FIG. 10) shows the decreasing amplitude of the sinusoidally varying radial cells deviation as induced by gravitational sedimentation. FIG. 10 also shows increasing radial cell deviation (per revolution) due to centrifugation as RPM is increased. These opposing constraints influence carefully choosing the optimal RPM to preferably minimize cell impact with, or accumulation at, the chamber walls. A family of curves is generated which is increasingly restrictive, in terms of workable RPM selections, as the external gravity field strength is increased or the cell sedimentation rate is increased. This family of curves, or preferably the computer model which solves these governing orbit equations, is preferably utilized to select the optimal RPM and chamber dimensions for the expansion of cells of a given sedimentation rate in a given external gravity field strength. Not to be bound by theory, but as a typical three-dimensional culture is expanded the number of cells and therefore the cell density effects the sedimentation rate, and therefore, the rotation rate may preferably be adjusted to optimize the same.

In the three-dimensional culture, the cell orbit (FIG. 9) from the rotating reference frame of the culture medium is seen to move in a nearly circular path under the influence of the rotating gravity vector (FIG. 11). Not to be bound by theory, but the two pseudo forces, coriolis and centrifugal, result from the rotating (accelerated) reference frame and cause distortion of the otherwise nearly circular path. Higher gravity levels and higher cell sedimentation rates produce larger radius circular paths which correspond to larger trajectory deviations from the ideal circular orbit as seen in the non-rotating reference frame. In the rotating reference frame it is thought, not to be bound by theory, that cells of differing sedimentation rates will remain spatially localized near each other for long periods of time with greatly reduced net cumulative separation than if the gravity vector were not rotated; the cells are sedimenting, but in a small circle (as observed in the rotating reference frame). Thus, in operation the present invention provides cells of differing sedimentation properties with sufficient time to interact mechanically and through soluble chemical signals thereby effecting their cell-to-cell interactions including geometry and support. In operation, the present invention provides for sedimentation rates of preferably from about 0 cm/second up to 10 cm/second.

Furthermore, in operation the culture chamber of the present invention has at least one aperture preferably for the input of fresh culture medium and a cell mixture and the removal of a volume of spent culture medium containing metabolic waste, but not limited thereto. Preferably, the exchange of culture medium can also be via a culture medium loop wherein fresh or recycled culture medium may be moved within the culture chamber preferably at a rate sufficient to support metabolic gas exchange, nutrient delivery, and metabolic waste product removal. This may slightly degrade the otherwise quiescent three-dimensional culture. It is preferable, therefore, to introduce a mechanism for the support of preferred components including, but not limited to, respiratory gas exchange, nutrient delivery, growth factor delivery to the culture medium of the three-dimensional culture, and also a mechanism for metabolic waste product removal in order to provide a long term three-dimensional culture able to support significant metabolic loads for periods of hours to months.

It is expected that expansion in a rotating bioreactor provides a unique environment that effects the cell phenotype, as gauged by RNA expression levels. The cells adapt to the unique three-dimensional environment in which they are suspended. Cells expanded in the three-dimensional environment of a rotating bioreactor express different gene expression patterns, and therefore, different membrane and surface protein configurations, and different cytoskeletal details. This feature of the cells is a result of the three-dimensional environment in which the cells are suspended and expanded, referred to as genetic expression modified. It is expected that the cell exposure to TVEMF in a TVEMF-bioreactor provides even more exceptional characteristics to the expanding peripheral blood cell than those detected by rotation alone.

During the time that the cells are in the rotating bioreactor (with or without TVEMF), they are preferably fed nutrients and fresh media (DMEM and 5% human serum albumin), exposed to hormones, cytokines, and/or growth factors (preferably G-CSF); and toxic materials are removed. The toxic materials removed from peripheral blood cells in a bioreactor include the toxic granular material of dying cells and the toxic material of granulocytes and macrophages.

Preferably, expansion is carried out in a rotating bioreactor at a temperature of about 26 C to about 41 C, and more preferably, at a temperature of about 37C.

One method of monitoring the overall expansion of cells undergoing expansion is by visual inspection. Peripheral blood stem cells are typically dark red in color. Once the bioreactor begins to rotate, and in a preferred embodiment the TVEMF is applied, the cells that are distributed throughout the full volume of media preferably cluster in the center of the bioreactor vessel as they become greater in number (denser), with the medium surrounding the colored cluster of cells. Oxygenation and other nutrient additions often do not cloud the ability to visualize the cell cluster through a visualization (typically clear plastic) window built into the bioreactor. Formation of the cluster is important for helping the stem cells maintain their three-dimensional geometry and cell-to-cell support and cell-to-cell geometry; if the cluster appears to scatter and cells begin to contact the wall of the bioreactor vessel, the rotational speed is increased (manually or automatically) so that the centralized cluster of cells may form again. A measurement of the visible diameter of the cell cluster taken soon after formation may be compared with later cluster diameters, to indicate the approximate number increase in cells in the bioreactor. Measurement of the increase in the number of cells during expansion may also be taken in a number of ways, as known in the art. An automatic sensor could also be included in the bioreactor to monitor and measure the increase in cluster size.

The expansion process may be carefully monitored, for instance by a laboratory expert, who will check cell cluster formation to ensure the cells remain clustered inside the bioreactor and will increase the rotation of the bioreactor when the cell cluster begins to scatter. An automatic system for monitoring the cell cluster and viscosity of the peripheral blood mixture inside the bioreactor may also monitor the cell clusters. A change in the viscosity of the cell cluster may become apparent about 2 days after beginning the expansion process, and the rotational speed of the bioreactor may be increased around that time. The bioreactor speed may vary throughout expansion. Preferably, the rotational speed is timely adjusted so that the cells undergoing expansion do not contact the sides of the rotating bioreactor vessel.

Also, the laboratory expert may, for instance once a day, or once every two days, manually (for instance with a syringe) insert fresh media and preferably other desired additives such as nutrients and growth factors, as discussed above, into the bioreactor, and draw off the old media containing cell wastes and toxins. Also, fresh media and other additives may be automatically pumped into the bioreactor during expansion, and wastes automatically removed.

Peripheral blood stem cells may increase to at least seven times their original number about 7 to about 14 days after being placed in the bioreactor and expanded. Preferably, the expansion lasts about 7 to 10 days, and more preferably about 7 days. Measurement of the number of stem cells does not need to be taken during expansion therefore. As indicated above and throughout this application, expanded peripheral blood stem cells of the present invention have essentially the same three-dimensional geometry and cell-to-cell support and cell-to-cell geometry as naturally-occurring, non-expanded peripheral blood stem cells due to the essentially non-turbulent and low shear stress culture regime. The expanded peripheral blood cell retains fundamental properties of the non-expanded peripheral blood cells. The gentle free drifting of the cells through soluble molecular species which control cell function and are substrates and products of cell metabolism allows the rotating bioreactor systems to produce a unique living product cell in terms of transcribed RNA pattern coding for multiple cell structural and functional proteins and cell sub organelles.

Another embodiment of the present invention relates to an ex vivo mammalian peripheral blood stem cell composition that functions to assist a body system or tissue to repair, replenish and regenerate tissue, for example, the tissues described throughout this application. The composition comprises expanded peripheral blood stem cells, preferably with TVEMF. The peripheral blood cells in the composition are preferably expanded to at least seven times the number that were placed in the culture chamber of the rotatable bioreactor. For instance, preferably, if a number X of peripheral blood stem cells was placed in a certain volume into a bioreactor, then after expansion, the number of peripheral blood stem cells from that same volume of peripheral blood stem cells place into the bioreactor will be at least 7×. While this at-least-seven-times-expansion is not necessary for this invention to work, this expansion is preferred for therapeutic purposes. For instance, the expanded cells may be only in amount of 2 times the number of peripheral blood stem cells placed in the rotating bioreactor, if desired. Preferably, expanded cells are in a range of about 4 times to about 25 times the number of peripheral blood stem cells placed in the bioreactor. In another preferred embodiment, the expanded cells number in an amount that is at least one cell more than the number that were placed in the culture chamber of the rotatable bioreactor. In this embodiment, the phenotypic expression of the cells after expansion is the preferred focus for repairing a body function or tissue.

The present invention is also directed to a composition comprising peripheral blood stem cells from a mammal, wherein said peripheral blood stem cells are expanded in a rotating TVEMF-bioreactor while suspending the cells therein to up or down regulate genes as effected by the cells environment, interactions, and three-dimensional geometry. A composition of the present invention may include a pharmaceutically acceptable carrier; plasma, peripheral blood, albumin, cell culture medium, growth factor, copper chelating agent, hormone, buffer or cryopreservative. “Pharmaceutically acceptable carrier” means an agent that will allow the introduction of the stem cells into a mammal, preferably a human. Such carrier may include substances mentioned herein, including in particular any substances that may be used for peripheral blood transfusion, for instance peripheral blood, plasma, albumin, preferably from the mammal to which the composition will be introduced. The term “introduction” of a composition to a mammal is meant to refer to “administration” of a composition to an animal. “Acceptable carrier” generally refers to any substance the peripheral blood stem cells of the present invention may survive in, ie that is not toxic to the cells, whether after TVEMF-expansion, prior to or after cryopreservation, prior to introduction (administration) into a mammal. Such carriers are well known in the art, and may include a wide variety of substances, including substances described for such a purpose throughout this application. For instance, plasma, peripheral blood, albumin, cell culture medium, buffer and cryopreservative are all acceptable carriers of this invention. The desired carrier may depend in part on the desired use.

Expanded peripheral blood stem cells have essentially the same, or maintain, the three-dimensional geometry and the cell-to-cell support and cell-to-cell geometry as the peripheral blood from which they originated. A preferred composition comprises expanded peripheral blood stem cells, preferably in a suspension of Dulbecco's medium or in a solution ready for cryopreservation. The composition is preferably free of toxic granular material, for example, dying cells and the toxic material or content of granulocytes and macrophages. The composition may be a cryopreserved composition comprising expanded peripheral blood stem cells by decreasing the temperature of the composition to a temperature of from −120° C. to −196° C. and maintaining the cryopreserved composition at that temperature range until needed for therapeutic or other use. As discussed below, preferably, as much toxic material as is possible is removed from the composition prior to cryopreservation.

Another embodiment of the present invention relates to a method of regenerating tissue and/or function with a composition of expanded peripheral blood stem cells, either having undergone cryopreservation or soon after expansion is complete. The cells may be introduced into a mammalian body, preferably human, for instance injected intravenously, directly into the tissue to be repaired, into the abdominal cavity, attaching to the peritoneum/peritoneal cavity, allowing the body's natural system to repair and regenerate the tissue. Preferably, the composition introduced into the mammalian body is free of toxic material and other materials that may cause an adverse reaction to the administered expanded peripheral blood stem cells.

An expanded peripheral blood stem cell composition of the present invention should be introduced into a mammal, preferably a human, in an amount sufficient to achieve repair of tissue and/or function, or to treat a desired disease or condition. Preferably, at least 20 ml of an expanded peripheral blood stem cell composition having 10⁷ to 10⁹ stem cells per ml is used for any treatment, preferably all at once, in particular where a traumatic injury has occurred and immediate tissue repair needed. This amount is particularly preferred in a 75-80 kg human. The amount of expanded peripheral blood stem cells in a composition being introduced into the source mammal is inherently related to the number of cells present in the source peripheral blood material. A preferred range of expanded peripheral blood stem cells introduced into a patient may be, for instance, about 10 ml to about 50 ml of an expanded peripheral blood stem cell composition having 10⁷ to 10⁹ stem cells per ml, or potentially even more. While it is understood that a high concentration of any substance, administered to a mammal, may be toxic or even lethal, it is unlikely that introducing all of a mammal's peripheral blood stem cells, for instance after expansion, will cause an overdose in expanded peripheral blood stem cells. Where peripheral blood from several donors is used, the number of peripheral blood stem cells introduced into a mammal may be higher. Therefore, it should be realized that the expanded cells may be introduced to the mammal from an allogeneic source or an autologous source. Also, the dosage of cells that may be introduced to the patient is not limited by the amount of peripheral blood provided from collection from one individual; multiple administrations, for instance once a day or twice a day, or once a week, or other administration time frames, may more easily be used. Also, where a tissue is to be treated, the type of tissue may warrant the use of as many expanded peripheral blood stem cells as are available.

### Example #1

### Qualitative and Quantitative Comparison Between a Rotating Bioreactor and a Dynamic Moving Culture

An experiment was conducted to demonstrate the qualitative differences between two cultures and the differences in the rates of expansion. To illustrate the differences a comparison was made between gene expression levels as assayed by abundance of mRNA transcripts in two samples of blood stem cells cultured in two different methods: (A) shaken Petri plate (dynamic moving culture) (B) rotating bioreactor. The cultures were set up, refed, harvested and otherwise manipulated in the identical manner. The test was documented using techniques well accepted in the art including Affymetrix Gene Array to prove the differences in genetic expression levels. All conditions and manipulations were the same for the two cultures except for the type of culture vessel in which they were expanded.

Culture A serves as the baseline on which to determine increase or decrease of transcript levels in culture B. There are several differences in membrane composition between the 2 cultures, as far as cell surface receptors are concerned. In addition, several of the other genes that are altered in the rotating bioreactor culture (mostly the ‘decreased’ ones) have a role in innate and adaptive immunity. Also, some transcripts of genes involved in cell-to-cell contacts and cytoskeletal structures are significantly changed. Some of the altered genes are involved in cell proliferation.

Below is a summary of the most relevant functions of a subset of the array data. Included in this summary are only those genes that show at least a 200% (1-fold) difference in expression levels between samples, either decreased (I) or increased (II). The data are further clustered based on cellular localization and/or function.

“Decreased” Genes (Range of Change is 4-to-1 Fold)

A. Membrane Proteins

1. Receptors

IL2R: aka CD25, expressed in regulatory T cells and macrophages and activated T- and B-cells; involved in cytokine-cytokine receptor interactions and role in cell proliferation

IL17R: receptor for IL17, and essential cytokine that acts as an immune response modulator

EV127: truncated precursor of IL17 receptor homolog

TGFR3: (aka beta-glycan) also has a soluble form; involved in cell differentiation, cell cycle progression, migration, adhesion, ECM production

FCGR1a: (aka CD64, human Fc-receptor) expressed in macrophages/monocytes, neutrophils; involved in phagocytosis, the immune response and cell signal transduction

MRC1: (aka CD206; Mannose Receptor; lectin-family) expressed in macrophages/monocytes (where expression increasing during culture), and dendritic cells; involved in innate and adaptive immunity

CCR1: (chemokine receptor, aka CD191, MIP1 receptor, RANTES receptor); multipass protein expressed in several hematopoietic cells that transduces a signal in response to several chemokines by increasing intracellular calcium ions level; responsible for affecting stem cell proliferation; role in cell adhesion, inflammation and immune response

CRL4: putative cytokine receptor precursor with role in signal transduction and proliferation

FERlL3: (myoferlin) single-pass protein at nuclear and plasma membranes; involved in membrane regeneration and repair; expressed in cardiac and skeletal muscle

EMP1: (aka TMP) multi-pass protein of claudin family involved in formation of tight junctions, and cell-to-cell contact

THBD: (thrombomodulin aka CD141); single pass endothelial cell receptor with lectin and EGF-like domains; complexes with thrombin to activate the coagulation cascade (factor Va and VIIIa)

2. Transporters

ABCA1: multipass protein involved in cholesterol trafficking (efflux); expressed in macrophages and keratinocytes

ABCG1: multi-pass transporter involved in macrophage lipid homeostasis; expressed in intracellular compartments of macrophages mostly; found in the endoplasmic reticulum membrane and Golgi apparatus;

3. Glycoproteins/Cell Surface

Versican (aka CSPG2, chondroitin sulfate proteoglycan 2); involved in maintaining ECM integrity, and has a role in cell proliferation, migration, and cell-cell adhesion (also interacts with tenascinR)

CD1c: expressed in activated Tcells; involved in mounting immune response

CD14: cell surface marker expressed in monocytes/macrophages

AREG: (amphiregulin) involved in cell-to-cell signaling and proliferation; growth-modulating glycoprotein. Inhibits growth of several human carcinoma cells in culture and stimulates proliferation of human fibroblasts and certain other tumor cells

Z39Ig: a membrane spanning immunoglobulin with a role in mounting the immune response; expressed in monocytes and dendritic cells

HML2: (aka CLEC10A, CD301) single pass lectin expressed in macrophages; Probable role in regulating adaptive and innate immune responses. Binds in a calcium-dependent manner to terminal galactose and N-acetylgalactosamine units, linked to serine or threonine.

CLECSF5: single pass myeloid lectin; involved in proinflammatory activation of myeloid cells via TYROBP-mediated signaling in a calcium-dependent manner

B. Cytosolic/Signal Transduction:

SKG1: expressed in granulocytes; has a role in response to oxidative stress and in cellular communication; part of the proteasome—ubiquitin pathway

C. Secreted

SCYA3 (aka CCL3, MIP1): secreted by macrophages/monocytes; soluble monokine with inflammatory and chemokinetic properties involved in mediating the inflammatory response; a major HIV-suppressive factor produced by CD8+ T-cells.

GRO3: (aka CKCL3, MIP2); secreted by PB monocytes; chemokine with chemotactic activity for neutrophils and a role in inflammation and immunity

Galectin3: soluble protein secreted by macrophages/monocytes; can bind the ECM to activate cells or restrain mobility; involved in other processes including inflammation, neoplastic transformation, and innate and acquired immunity by binding IgE; also has a nuclear form; inhibited by MMP9.

D. Nuclear/Transcription Factors

KRML; LOC51713; KLF4: three gene members of Kreisler/Krox family of nuclear transcription factors involved in bone and inner ear morphogenesis, epithelial cell differentiation and/or development of the skeleton and kidney

EGR1: (aka KROX24) expressed in lymphocytes and lymphoid organs; involved in macrophage differentiation, and inflammation/apoptosis pathways; activates genes in differentiation

E. Enzymes

HMOX1: (heme oxygenase) microsomal (ER); highly expressed in spleen; involved in heme turnover; ubiquitously expressed following induction by several stresses, potent anti-inflammatory proteins whenever oxidation injury takes place

BPHL: mitochondrial serine hydrolase that catalyzes the hydrolytic activation of amino acid ester prodrugs of nucleoside analogs; may play a role in detoxification processes

“Increased” Genes (Range of Change is 2-to-1 Fold)

A. Membrane Proteins

Proteoglycan 3: expressed in eosinophils and granulocytes, highly expressed in bone marrow; involved in immune response, neutrophil activation and release of IL8 and histamine

CYP1B1: Cytochromes P450 are a group of heme-thiolate monooxygenases involved in an NADPH-dependent electron transport pathway. It oxidizes a variety of structurally unrelated compounds, including steroids, fatty acids, and xenobiotics

IL9R: single pass interleukin receptor, involved in cell proliferation and signaling, expressed in hematopoietic cells

HBA1: (CD31) binds heme and iron involved in oxygen transport, specific to RBCs

RHAG (aka CD241) expressed in erythrocytes, Rh blood group protein multipass protein ammonium transporter; binds ankyrin, a component of the RBC cytoskeleton

B. Cytoskeletal Proteins

SPTA1; ANK1: both proteins are located on cytoplasmic face of plasma membrane of erythrocytes (RBC) and act to anchor transmembrane proteins to the cytoskeleton; together with actin and other proteins they form the RBC cytoskeleton superstructure and are responsible for keeping its shape

NCALD: neurocalcin; cytosolic; involved in vesicle-mediated transport; binds actin, tubulin and clathrin; can bind Ca2+; expressed in neural tissues and testes

C. Enzymes (Cytosolic)

LSS: cholesterol metabolism-steroid biosynthesis

PDE4B: involved in anti-inflammatory response, high in CNS; purine metabolism

SPUVE: a secreted serine protease (unknown function)

ELA2: serine protease expressed in leukocytes/neutrophils, involved in protein hydrolysis including elastin; serves to modify the function of NK cells, monocytes and granulocytes; inhibits chemotaxis in anti-inflammatory response, high in BM

HGD: iron binding oxygenase involved in tyrosine metab and phenylalanine catabolism

ADAMDEC1: expressed in macrophages; a secreted zinc binding serum protease involved in immune response; up-regulated during primary monocyte to macrophage and/or dendritic cell differentiation

HMGCS1: soluble co-enzyme A synthase involved in cholesterol biosynthesis

COVA1 hydroquinone oxidase (X-linked) extracellular and trans plasma membrane associated (secreted factor) has copper as a cofactor has several properties associated with prions; naturally is glycosylated; involved ultradian rhythm maintenance, cell growth regulation, electron transport

PFKB4: glycolytic enzyme

D. Nuclear/Transcription Factors

Pirin: iron-binding nuclear transcription factor; DNA replication and transactivation (X-linked); interacts with SMAD signaling cascade

E. Other

S100A8, A9: secreted, calcium binding proteins (isoforms A8, A9 expressed in epithelial cells) expressed by monocytes/macrophages and granulocytes as part of the inflammatory response; inhibitor of protein kinases. Also expressed in epithelial cells constitutively or induced during dermatoses. May interact with components of the intermediate filaments in monocytes and epithelial cells; highly expressed in bone marrow.

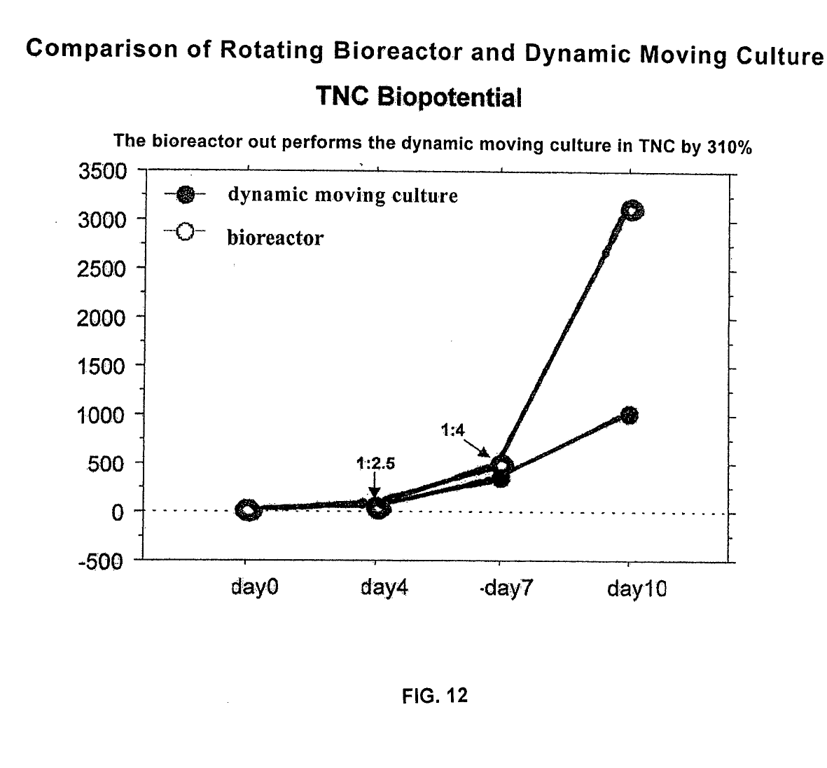

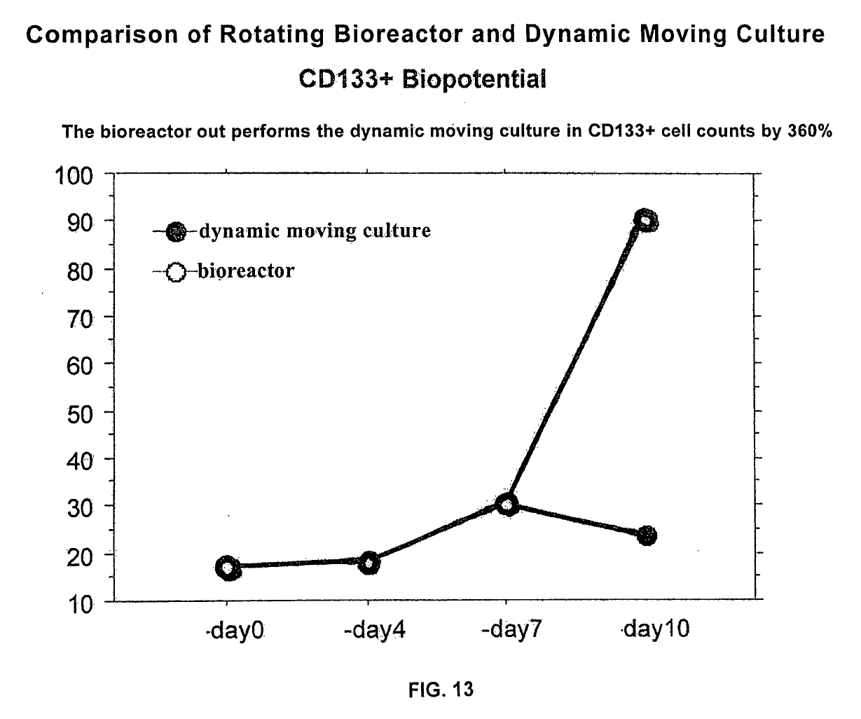

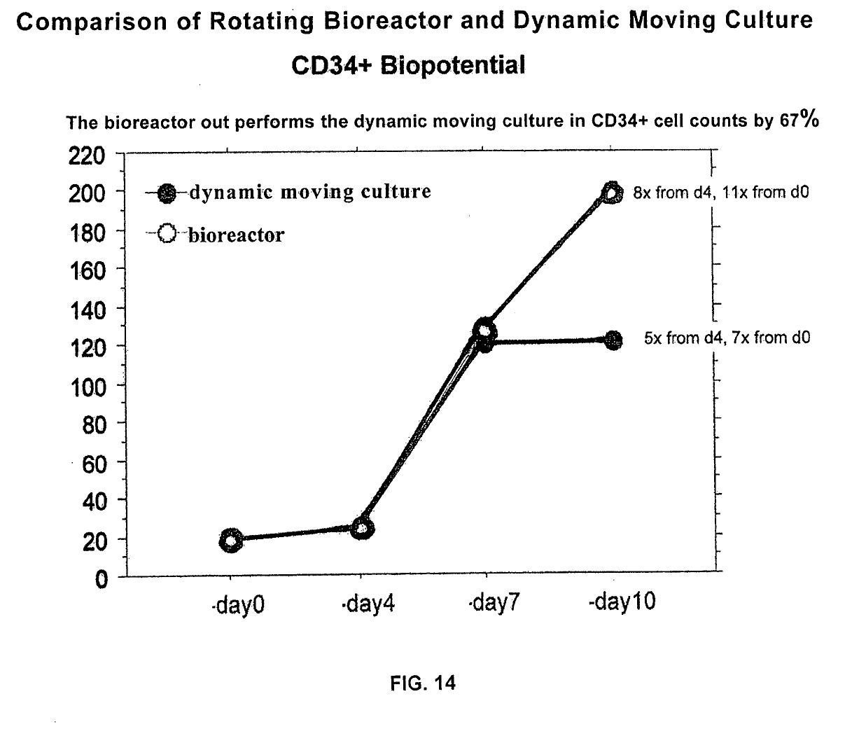

FIGS. 12, 13, and 14 illustrate that the cells in a rotating bioreactor expand to a significantly greater number than cells in a dynamic moving culture. The expansion of CD133+ cells, total nucleated cells and CD34+ cells were analyzed.

These results demonstrate that cells expanded in a rotating system, such as a TVEMF-bioreactor are qualitatively unique. The non-turbulent regime in the rotating bioreactor allows the cells to expand in a low shear environment so that the input cell is not disturbed as much as it would be in other three-dimensional systems. However, as a result of the expansion process, the expanded blood stem cells have a unique phenotypic expression to support their suspension in the three-dimensional environment. That expression is fostered and maintained without differentiation and over a high rate of expansion.

### Example #2

### TVEMF-Expansion in a TVEMF-Bioreactor

CD133-selected cells were pre-cultured in a two-dimensional culture system for three days prior to placing the cells in a rotating bioreactor with and without TVEMF. Samples V1 and V2 were cultured without TVEMF and V1T and V2T were cultured with TVEMF, while all other conditions stayed the same. The cells were placed in a 10 ml rotating TVEMF-bioreactor at a density of about 0.2×10⁶ cells/ml, and the entire bioreactor volume was filled. The culture medium used for this experiment was IMDM. The bioreactors were rotated at approximately 20 rpm. The following data refers to the culture period in the rotating TVEMF-bioreactor, and does not reflect the two-dimensional pre-culture. The cultures were expanded at 37° C., and in 5% CO₂. All other culture conditions remained the same for each sample, V1, V2, V1T and V2T.

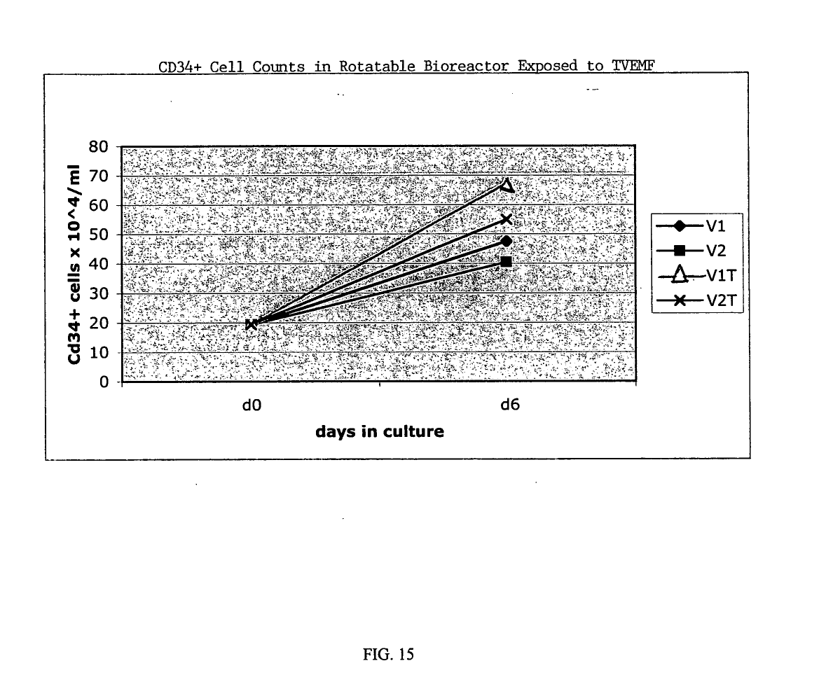

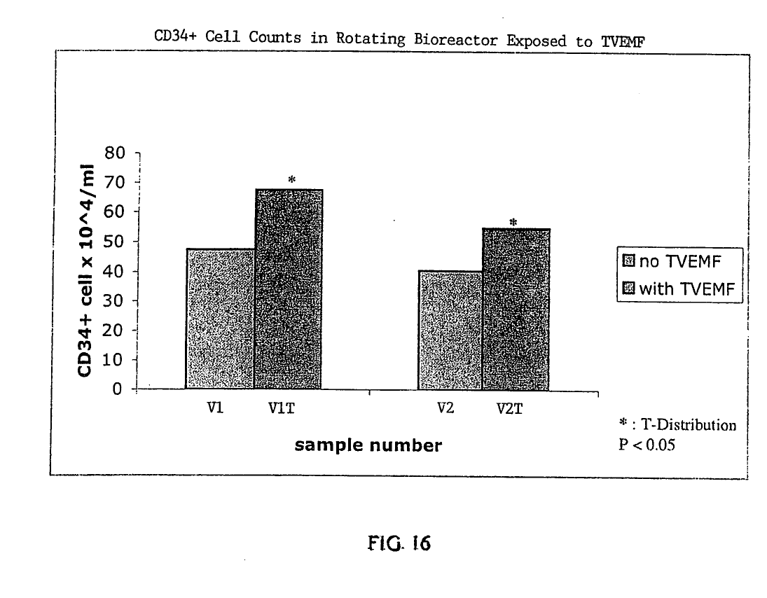

FIG. 15 illustrates the results of the TVEMF-expansion (numbers of cells). The number of CD34+ cells increased from between 20×10⁴ cells/ml and 48×10⁴ cells/ml by day 6. FIG. 16 illustrates the expansion rate (number of cells) in a rotating TVEMF-bioreactor as compared with a rotating non-TVEMF bioreactor. The results show that on day 6, the cultures that were exposed to TVEMF had more cells than those that were not. The difference between expansion with and without TVEMF was between about 10×10⁴ cells/ml and about 15×10⁴ cells/ml.

### Example #3

### TVEMF-Expansion of Cells in a TVEMF Bioreactor

Peripheral blood was collected and peripheral blood cells expanded as shown in Table 1, and described below.

A) Collection and Maintenance of Cells

Human peripheral blood (75 ml; about 0.75×10⁶ cells/ml) was collected from 15 human donors by syringe as above; blood collected from 10 donors was suspended in 75 ml Iscove's modified Dulbecco's medium (IMDM) (GIBCO, Grand Island, N.Y.) supplemented with 20% of 5% human albumin (HA), 100 ng/ml recombinant human G-CSF (Amgen Inc., Thousand Oaks, Calif.), and 100 ng/mil recombinant human stem cell factor (SCF) (Amgen) to prepare a peripheral blood mixture. Part of each peripheral blood sample was set aside as a “control” sample. The peripheral blood mixture was placed in a TVEMF-bioreactor as shown in FIGS. 2 and 3 herein. TVEMF-expansion occurred at 37° C., 6% CO₂, with a normal air O₂/N ratio. The TVEMF-bioreactor was rotated at a speed of 10 rotations per minute (rpm) initially, and adjusted as needed, as described throughout this application, to keep the peripheral blood cells suspended in the bioreactor. A time varying current of 6 mA was applied to the bioreactor. The square wave TVEMF signal applied to the peripheral blood mixture was about 0.5 Gauss. (frequency: about 10 cycles/sec). Culture media in the peripheral blood mixture in the TVEMF-bioreactor was changed/freshened every one to two days. At day 10, the cells were removed from the TVEMF-bioreactor and washed with PBS and analyzed. The results are as set forth in Table 1. Control data refers to a sample of human peripheral blood that has not been expanded; Expanded Sample refers to the respective control sample after TVEMF-expansion.

TABLE 1

| | | |
|---|---|---|
| Control 1 | Cell Count 300,000 | Viability 98% |
| Control 2 | Cell Count 325,000 | Viability 100% |
| Control 3 | Cell Count 350,000 | Viability 98% |
| Control 4 | Cell Count 300,000 | Viability 98% |
| Control 5 | Cell Count 315,000 | Viability 99% |
| Control 6 | Cell Count 320,000 | Viability 98% |
| Control 7 | Cell Count 310,000 | Viability 98% |
| Control 8 | Cell Count 340,000 | Viability 100% |
| Control 9 | Cell Count 300,000 | Viability 98% |
| Control 10 | Cell Count 320,000 | Viability 98% |
| Expanded Sample 1 | Cell Count 3,000,000 | Viability 99% |
| | Corresponding CD34+ | |
| | increase: yes | |
| Expanded Sample 2 | Cell Count 3,500,000 | Viability 100% |
| | Corresponding CD34+ | |
| | increase: yes | |
| Expanded Sample 3 | Cell Count 3,750,000 | Viability 98% |
| | Corresponding CD34+ | |
| | increase: yes | |
| Expanded Sample 4 | Cell Count 3,250,000 | Viability 98% |
| | Corresponding CD34+ | |
| | increase: yes | |
| Expanded Sample 5 | Cell Count 3,450,000 | Viability 100% |
| | Corresponding CD34+ | |
| | increase: yes | |
| Expanded Sample 6 | Cell Count 3,400,000 | Viability 98% |
| | Corresponding CD34+ | |
| | increase: yes | |
| Expanded Sample 7 | Cell Count 3,200,000 | Viability 98% |
| | Corresponding CD34+ | |
| | increase: yes | |
| Expanded Sample 8 | Cell Count 3,500,000 | Viability 100% |
| | Corresponding CD34+ | |
| | increase: yes | |
| Expanded Sample 9 | Cell Count 3, 150,000 | Viability 98% |
| | Corresponding CD34+ | |
| | increase: yes | |
| Expanded Sample 10 | Cell Count 3,500,000 | Viability 99% |
| | Corresponding CD34+ | |
| | increase: yes | |

As may be seen from Table 1, TVEMF-expansion of peripheral blood cells resulted in roughly a 10-fold increase in the number of cells over 10 days, as compared to non-expanded control, with a corresponding increase in CD34+ cells. The culture media where the cells were growing was changed/freshened once every 1-2 days.

B) Analysis of TVEMF-Expanded Cells

Total cell counts of Control and Expanded Samples were obtained with a counting chamber (a device such as a hemocytometer used by placing a volume of either the control cell suspension or expanded sample on a specially-made microscope slide with a microgrid and counting the number of cells in the sample). The results of the total cell counts in Control samples and in Expanded Samples after 10 days of TVEMF-expansion are shown in Table 1.

The indication of corresponding CD34+ increase in Table 1 was determined as follows: CD34+ cells of the Expanded Samples were separated from other cells therein with a Human CD34 Selection Kit (EasySep positive selection, StemCell Technologies), and counted with a counting chamber as indicated above and confirmed with FACScan flow cytometer (Becton-Dickinson). CFU-GEMM and CFU-GM were counted by clonogenic assay. Cell viability (where a viable cell is alive and a non-viable cell is dead) was determined by trypan blue exclusion test. The answer of “yes” in all Expanded Samples indicates that the number of CD34+ cells increased in amounts corresponding to the total cell count.

C) Increase in Amount of Hematopoietic Colony-Forming Cells

Incubation of the donors' peripheral blood cells in this TVEMF-expansion tissue culture system significantly increases the numbers of hematopoietic colony-forming cells. As determined in a separate assay, a constant increase in the numbers of CFU-GM (up to 7-fold) and CFU-GEMM (up to 9-fold) colony-forming cells is observed up to day 7 with no clear plateau.

D) Increase in CD34+ Cells

Incubation of MNCs from normal donors in this TVEMF-expansion tissue culture system significantly increases the numbers of CD34+ cells. As determined in a separate assay, the average number of CD34+ cells increased 10-fold by day 6 of culture and plateaus on that same day.

### Operative Method

### Cryopreservation

As mentioned above, peripheral blood is collected from a mammal, preferably a human. Red blood cells, at least, are preferably removed from the peripheral blood. The peripheral blood stem cells (with other cells and media as desired) are placed in a bioreactor, preferably a TVEMF-bioreactor and subjected to a time varying electromagnetic force, and expanded. If RBCs were not removed prior to expansion, preferably they are removed after expansion. The expanded cells may be cryogenically preserved. Further details relating to a method for the cryopreservation of expanded peripheral blood stem cells, and compositions comprising such cells are provided herein and in particular below.

After, for instance, TVEMF-expansion, the TVEMF-expanded cells, including TVEMF-expanded peripheral blood stem cells, are preferably transferred into at least one cryopreservation container containing at least one cryoprotective agent. The TVEMF-expanded peripheral blood stem cells are preferably first washed with a solution (for instance, a buffer solution or the desired cryopreservative solution) to remove media and other components present during TVEMF-expansion, and then preferably mixed in a solution that allows for cryopreservation of the cells. Such solution is commonly referred to as a cryopreservative, cryopreservation solution or cryoprotectant. The cells are transferred to an appropriate cryogenic container and the container decreased in temperature to generally from −120° C. to −196° C., preferably about −130° C. to about −150° C., and maintained at that temperature. Preferably, this decrease in temperature is done slowly and carefully, so as to not damage, or at least to minimize damage, to the stem cells during the freezing process. When needed, the temperature of the cells (about the temperature of the cryogenic container) is raised to a temperature compatible with introduction of the cells into the human body (generally from around room temperature to around body temperature), and the TVEMF-expanded cells may be introduced into a mammalian body, preferably human, for instance as discussed throughout this application.

Freezing cells is ordinarily destructive. Not to be bound by theory, on cooling, water within the cell freezes. Injury then may occur by osmotic effects on the cell membrane, cell dehydration, solute concentration, and ice crystal formation. As ice forms outside the cell, available water is removed from solution and withdrawn from the cell, causing osmotic dehydration and raised solute concentration that may eventually destroy the cell. (For a discussion, see Mazur, P., 1977, Cryobiology 14:251-272.)

Different materials have different freezing points. Preferably, a peripheral blood stem cell composition ready for cryopreservation contains as few contaminating substances as possible, to minimize cell wall damage from the crystallization and freezing process.

These injurious effects can be reduced or even circumvented by (a) use of a cryoprotective agent, (b) control of the freezing rate, and (c) storage at a temperature sufficiently low to minimize degradative reactions.

The inclusion of cryopreservation agents is preferred in the present invention. Cryoprotective agents which can be used include but are not limited to a sufficient amount of dimethyl sulfoxide (DMSO) (Lovelock, J. E. and Bishop, M. W. H., 1959, Nature 183:1394-1395; Ashwood-Smith, M. J., 1961, Nature 190:1204-1205), glycerol, polyvinylpyrrolidine (Rinfret, A. P., 1960, Ann. N.Y. Acad. Sci. 85:576), polyethylene glycol (Sloviter, H. A. and Ravdin, R. G., 1962, Nature 196:548), albumin, dextran, sucrose, ethylene glycol, i-erythritol, D-ribitol, D-mannitol (Rowe, A. W., et al., 1962, Fed. Proc. 21:157), D-sorbitol, i-inositol, D-lactose, choline chloride (Bender, M. A., et al., 1960, J. Appl. Physiol. 15:520), amino acid-glucose solutions or amino acids (Phan The Tran and Bender, M. A., 1960, Exp. Cell Res. 20:651), methanol, acetamide, glycerol monoacetate (Lovelock, J. E., 1954, Biochem. J. 56:265), and inorganic salts (Phan The Tran and Bender, M. A., 1960, Proc. Soc. Exp. Biol. Med. 104:388; Phan The Tran and Bender, M. A., 1961, in Radiobiology, Proceedings of the Third Australian Conference on Radiobiology, ilbery, P. L. T., ed., Butterworth, London, p. 59). In a preferred embodiment, DMSO is used. DMSO, a liquid, is nontoxic to cells in low concentration. Being a small molecule, DMSO freely permeates the cell and protects intracellular organelles by combining with water to modify its freezability and prevent damage from ice formation. Adding plasma (for instance, to a concentration of 20-25%) can augment the protective effect of DMSO. After addition of DMSO, cells should be kept at 0° C. or below, since DMSO concentrations of about 1% may be toxic at temperatures above 4° C. My selected preferred cryoprotective agents are, in combination with TVEMF-expanded peripheral blood stem cells for the total composition: 20 to 40% dimethyl sulfoxide solution in 60 to 80% amino acid-glucose solution, or 15 to 25% hydroxyethyl starch solution, or 4 to 6% glycerol, 3 to 5% glucose, 6 to 10% dextran T10, or 15 to 25% polyethylene glycol or 75 to 85% amino acid-glucose solution. The amount of cryopreservative indicated above is preferably the total amount of cryopreservative in the entire composition (not just the amount of substance added to a composition).

While other substances, other than peripheral blood cells and a cryoprotective agent, may be present in a composition of the present invention to be cryopreserved, preferably cryopreservation of a TVEMF-expanded peripheral blood stem cell composition of the present invention occurs with as few other substances as possible, for instance for reasons such as those discussed regarding the mechanism of freezing, above.

Preferably, expanded peripheral blood stem cell composition of the present invention is cooled to a temperature in the range of about −120° C. to about −196° C., preferably about −130° C. to about −196° C., and even more preferably about −130° C. to about −150° C.

A controlled slow cooling rate is critical. Different cryoprotective agents (Rapatz, G., et al., 1968, Cryobiology 5(1):18-25) and different cell types have different optimal cooling rates (see e.g. Rowe, A. W. and Rinfret, A. P., 1962, Blood 20:636; Rowe, A. W., 1966, Cryobiology 3(1):12-18; Lewis, J. P., et al., 1967, Transfusion 7(1):17-32; and Mazur, P., 1970, Science 168:939-949 for effects of cooling velocity on survival of peripheral cells (and on their transplantation potential)). The heat of fusion phase where water turns to ice should be minimal. The cooling procedure can be carried out by use of, e.g., a programmable freezing device or a methanol bath procedure.

Programmable freezing apparatuses allow determination of optimal cooling rates and facilitate standard reproducible cooling. Programmable controlled-rate freezers such as Cryomed or Planar permit tuning of the freezing regimen to the desired cooling rate curve. Other acceptable freezers may be, for example, Sanyo Modl MDF-1155ATN-152C and Model MDF-2136ATN-135C, Princeton CryoTech TEC 2000. For example, for peripheral blood cells or CD34+ cells in 10% DMSO and 20% plasma, the optimal rate is 1 to 3° C./minute from 0° C. to −200° C.

In a preferred embodiment, this cooling rate can be used for the cells of the invention. The cryogenic container holding the cells must be stable at cryogenic temperatures and allow for rapid heat transfer for effective control of both freezing and thawing. Sealed plastic vials (e.g., Nunc, Wheaton cryules) or glass ampules can be used for multiple small amounts (1-2 ml), while larger volumes (100-200 ml) can be frozen in polyolefin bags (e.g., Delmed) held between metal plates for better heat transfer during cooling. (Bags of bone marrow cells have been successfully frozen by placing them in −80° C. freezers that, fortuitously, gives a cooling rate of approximately 3° C./minute).

In an alternative embodiment, the methanol bath method of cooling can be used. The methanol bath method is well suited to routine cryopreservation of multiple small items on a large scale. The method does not require manual control of the freezing rate nor a recorder to monitor the rate. In a preferred aspect, DMSO-treated cells are precooled on ice and transferred to a tray containing chilled methanol that is placed, in turn, in a mechanical refrigerator (e.g., Harris or Revco) at −130° C. Thermocouple measurements of the methanol bath and the samples indicate the desired cooling rate of 1 to 3° C./minute. After at least two hours, the specimens will reach a temperature of −80° C. and may be placed directly into liquid nitrogen (−196° C.) for permanent storage.

After thorough freezing, expanded stem cells can be rapidly transferred to a long-term cryogenic storage vessel (such as a freezer). In a preferred embodiment, the cells can be cryogenically stored in liquid nitrogen (−196° C.) or its vapor (−165° C.). The storage temperature should be below −120° C., preferably below −130° C. Such storage is greatly facilitated by the availability of highly efficient liquid nitrogen refrigerators, which resemble large Thermos containers with an extremely low vacuum and internal super insulation, such that heat leakage and nitrogen losses are kept to an absolute minimum.

The preferred apparatus and procedure for the cryopreservation of the cells is that manufactured by Thermogenesis Corp., Rancho Cordovo, Calif., utilizing their procedure for lowering the cell temperature to below −130° C. The cells are held in a Thermogenesis plasma bag during freezing and storage.

Other freezers are commercially available. For instance, the “BioArchive” freezer not only freezes but also inventories a cryogenic sample such as peripheral blood or cells of the present invention, for instance managing up to 3,626 bags of frozen peripheral blood at a time. This freezer has a robotic arm that will retrieve a specific sample when instructed, ensuring that no other examples are disturbed or exposed to warmer temperatures. Other freezers commercially available include, but are not limited to, Sanyo Model MDF-1155 ATN-152C and Model MDF-2136 ATN-135C, and Princeton CryoTech TEC 2000.

After the temperature of the expanded peripheral blood stem cell composition is reduced to below −120° C., preferably below −130° C., they may be held in an apparatus such as a Thermogenesis freezer. Their temperature is maintained at a temperature of about −120° C. to −196° C., preferably −130° C. to −150° C. The temperature of a cryopreserved expanded peripheral blood stem cell composition of the present invention should not be above −120° C. for a prolonged period of time.

Cryopreserved expanded peripheral blood stem cells, preferably TVEMF-expanded peripheral blood stem cells, or a composition thereof, according to the present invention may be frozen for an indefinite period of time, to be thawed when needed. For instance, a composition may be frozen for up to 18 years. Even longer time periods may work, perhaps even as long as the lifetime of the peripheral blood donor.

When needed, bags with the cells therein may be placed in a thawing system such as a Thermogenesis Plasma Thawer or other thawing apparatus such as in the Thermoline Thawer series. The temperature of the cryopreserved composition is raised to room temperature. In another preferred method of thawing cells mixed with a cryoprotective agent, bags having a cryopreserved TVEMF-expanded peripheral blood stem cell composition of the present invention, stored in liquid nitrogen, may be placed in the gas phase of liquid nitrogen for 15 minutes, exposed to ambient air room temperature for 5 minutes, and finally thawed in a 37° C. water bath as rapidly as possible. The contents of the thawed bags may be immediately diluted with an equal volume of a solution containing 2.5% (weight/volume) human serum albumin and 5% (weight/volume) Dextran 40 (Solplex 40; Sifra, Verona, Italy) in isotonic salt solution and subsequently centrifuged at 400 g for ten minutes. The supernatant would be removed and the sedimented cells resuspended in fresh albumin/Dextran solution. See Rubinstein, P. et al., Processing and cryopreservation of placental/umbilical cord blood for unrelated bone marrow reconstitution. Proc. Natl. Acad. Sci. 92:10119-1012 (1995) for Removal of Hypertonic Cryoprotectant; a variation on this preferred method of thawing cells can be found in Lazzari, L. et al., Evaluation of the effect of cryopreservation on ex vivo expansion of hematopoietic progenitors from cord blood. Bone Marrow Trans. 28:693-698 (2001).

After the cells are raised in temperature to room temperature, they are available for research or regeneration therapy. The thawed expanded peripheral blood stem cell composition may be introduced directly into a mammal, preferably human, or used in its thawed form for instance for desired research. The solution in which the thawed cells are present may be completely washed away, and exchanged with another, or added to or otherwise manipulated as desired. Various additives may be added to the thawed compositions (or to a non-cryopreserved TVEMF-expanded peripheral blood stem cell composition) prior to introduction into a mammalian body, preferably soon to immediately prior to such introduction. Such additives include but are not limited to a growth factor, a copper chelating agent, a cytokine, a hormone, a suitable buffer or diluent. Preferably, G-CSF is added. Even more preferably, for humans, G-CSF is added in an amount of about 20 to about 40 micrograms/kg body weight, and even more preferably in an amount of about 30 micrograms/kg body weight. Also, prior to introduction, the TVEMF-expanded peripheral blood stem cell composition may be mixed with the mammal's own, or a suitable donor's, plasma, peripheral blood or albumin, or other materials that for instance may accompany peripheral blood transfusions. The thawed peripheral blood stem cells can be used for instance to test to see if there is an adverse reaction to a pharmaceutical that is desired to be used for treatment or they can be used for treatment.

While the FDA has not approved use of expanded peripheral blood stem cells for regeneration of tissue in the United States, such approval appears to be imminent. Direct injection of a sufficient amount of expanded peripheral blood stem cells should be able to be used to repair and regenerate tissue.

During the entire process of expansion, preservation, and thawing, blood stem cells of the present invention maintain the phenotypic characteristics maintained, fostered, and developed as a result of the expansion process and also TVEMF-expansion process.

An expanded, preferably TVEMF-expanded, blood stem cell composition of the present invention should be introduced into a mammal, preferably a human, in a “therapeutically effective” amount, sufficient to achieve tissue repair or regeneration, or to treat a desired disease or condition. Preferably, at least 20 ml of a TVEMF-expanded blood stem cell composition having 10⁷ to 10⁹ stem cells per ml is used for any treatment, preferably all at once, in particular where a traumatic injury has occurred and immediate tissue repair needed. This amount is particularly preferred in a 75-80 kg human. The amount of expanded blood stem cells in a composition being introduced into a mammal depends in part on the number of cells present in the source blood material (in particular if only a fairly limited amount is available). A preferred range of TVEMF-expanded blood stem cells introduced into a patient may be, for instance, about 10 ml to about 50 ml of a TVEMF-expanded blood stem cell composition having 10⁷ to 10⁹ stem cells per ml, or potentially even more. While it is understood that a high concentration of any substance, administered to a mammal, may be toxic or even lethal, it is unlikely that introducing all of the expanded blood stem cells, for instance after expansion at least 7 times, will cause an overdose in expanded blood stem cells. Where blood from several donors or multiple collections from the same donor is used, the number of blood stem cells introduced into a mammal may be higher. Also, the dosage of cells that may be introduced to the patient is not limited by the amount of blood provided from collection from one individual; multiple administrations, for instance once a day or twice a day, or once a week, or other administration time frames, may more easily be used. Also, where a tissue is to be treated, the type of tissue may warrant the use of as many expanded blood stem cells as are available, or the use of a smaller dose.

It is to be understood that, while the embodiment described above generally relates to cryopreserving expanded blood stem cells, expansion may occur after thawing of already cryopreserved, non-expanded, or non-TVEMF-expanded, blood stem cells. Also, if cryopreservation is desired, expansion may occur both before and after freezing the cells. Blood banks, for instance, have cryopreserved compositions comprising blood stem cells in frozen storage, in case such is needed at some point in time. Such compositions may be thawed according to conventional methods and then expanded as described herein, including variations in the process as described herein. Thereafter, such expanded peripheral blood stem cells are considered to be compositions of the present invention, as described above. Expansion prior to cryopreserving is preferred, for instance as if a traumatic injury occurs, a patient's peripheral blood stem cells have already been expanded and do not require precious extra days to prepare.

Also, while not preferred, it should be noted that expanded peripheral blood stem cells of the present invention may be cryopreserved, and then thawed, and then if not used, cryopreserved again. Prior to the cells being frozen, the cells are preferably TVEMF-expanded (that is, increased in number, not size). The cells may also be expanded after being frozen and then thawed, even if already expanded before freezing.

Expansion of peripheral blood stem cells may take several days. In a situation where it is important to have an immediate supply of peripheral blood stem cells, such as a life-or-death situation or in the case of a traumatic injury, especially if research needs to be accomplished prior to reintroduction of the cells, several days may not be available to await the expansion of the peripheral blood stem cells. It is particularly desirable, therefore, to have such expanded peripheral blood stem cells available from birth forward in anticipation of an emergency where every minute in delaying treatment can mean the difference in life or death.

Also, it is to be understood that the expanded peripheral blood stem cells of the present application may be introduced into a mammal, preferably the source mammal (mammal that is the source of the peripheral blood), after expansion, with or without cryopreservation. However, such introduction need not be limited to only the source mammal (autologous); the expanded cells may also be transferred to a different mammal (allogenic).

Also, it is to be understood that, while peripheral blood is the preferred source of adult stem cells for the present invention, adult stem cells from bone marrow may also be expanded, preferably TVEMF-expanded, and used in a manner similar to peripheral blood stem cells in the present invention. Bone marrow is not a readily available source of stem cells, but must be collected via apheresis or some other expensive and painful method.

The present invention also includes a method of researching tissue, for instance in relation to a disease or condition. The method may include, for instance, introducing a peripheral blood stem cell composition into a test system for the disease state. Such as system may include, but is not limited to, for instance a mammal having the disease, an appropriate animal model for studying the disease or an in vitro test system for studying the disease. Expanded and TVEMF-expanded peripheral blood stem cells may be used for research for possible cures for the following diseases:

I. Diseases resulting from a failure or dysfunction of normal blood cell production and maturation, hyperproliferative stem cell disorders, aplastic anemia, pancytopenia, thrombocytopenia, red cell aplasia, Blackfan-Diamond syndrome due to drugs, radiation, or infection, idiopathic;

II. Hematopoietic malignancies, acute lymphoblastic (lymphocytic) leukemia, chronic lymphocytic leukemia, acute myelogenous leukemia, chronic myelogenous leukemia, acute malignant myelosclerosis, multiple myeloma, polycythemia vera, agnogenic myelometaplasia, Waldenstrom's macroglobulinemia, Hodgkin's lymphoma, non-Hodgkins's lymphoma;

III. Immunosuppression in patients with malignant, solid tumors, malignant melanoma, carcinoma of the stomach, ovarian carcinoma, breast carcinoma, small cell lung, carcinoma, retinoblastoma, testicular carcinoma, glioblastoma, rhabdomyosarcoma, neuroblastoma, Ewing's sarcoma, lymphoma;

IV. Autoimmune diseases, rheumatoid arthritis, diabetes type I, chronic hepatitis, multiple sclerosis, and systemic lupus erythematosus;

V. Genetic (congenital) disorders, anemias, familial aplastic, Fanconi's syndrome, Bloom's syndrome, pure red cell aplasia (PRCA), dyskeratosis congenital, Blackfan-Diamond syndrome, congenital dyserythropoietic syndromes I-IV, Chwachmann-Diamond syndrome, dihydrofolate reductase deficiencies, formamino transferase deficiency, Lesch-Nyhan syndrome, congenital spherocytosis, congenital elliptocytosis, congenital stomatocytosis, congenital Rh null disease, paroxysmal nocturnal hemoglobinuria, G6PD (glucose-6-phosphate dehydrogenase), variants 1,2,3, pyruvate kinase deficiency, congenital erythropoietin sensitivity, deficiency, sickle cell disease and trait, thalassemia alpha, beta, gamma met-hemoglobinemia, congenital disorders of immunity, severe combined immunodeficiency disease, (SCID), bare lymphocyte syndrome, ionophore-responsive combined, immunodeficiency, combined immunodeficiency with a capping abnormality, nucleoside phosphorylase deficiency, granulocyte actin deficiency, infantile agranulocytosis, Gaucher's disease, adenosine deaminase deficiency, Kostmann's syndrome, reticular dysgenesis, congenital leukocyte dysfunction syndromes; and

VI. Others including osteopetrosis, myelosclerosis, acquired hemolytic anemias, acquired immunodeficiencies, infectious disorders causing primary or secondary immunodeficiencies, bacterial infections (e.g., Brucellosis, Listerosis, tuberculosis, leprosy), parasitic infections (e.g., malaria, Leishmaniasis), fungal infections, disorders involving disproportions in lymphoid cell sets and impaired immune functions due to aging phagocyte disorders, Kostmann's agranulocytosis, chronic granulomatous disease, Chediak-Higachi syndrome, neutrophil actin deficiency, neutrophil membrane GP-180 deficiency, metabolic storage diseases, mucopolysaccharidoses, mucolipidosis, miscellaneous disorders involving immune mechanisms, Wiskott-Aldrich Syndrome, alpha 1-antitrypsin deficiency.

During the entire process of expansion, preservation, and thawing, peripheral blood stem cells of the present invention maintain their unique phenotypic expression acquired, fostered, and maintained as a result of the TVEMF-expansion process.

While preferred embodiments have been herein described, those skilled in the art will understand the present invention to include various changes and modifications. The scope of the invention is not intended to be limited to the above-described embodiments.

I claim:

1. Time varying electromagnetic force (“TVEMF”) expanded peripheral blood stem cells from a mammal,
wherein the expanded peripheral blood stem cells have been TVEMF-expanded in a three-dimensional environment of a TVEMF-bioreactor rotating about its horizontal longitudinal central axis;
wherein the expanded peripheral blood stem cells are genetic expression modified by the TVEMF-expansion in the TVEMF bioreactor; and
wherein the cells are expanded in the TVEMF-bioreactor without substantial differentiation.

2. A composition comprising the peripheral blood stem cells of claim 1 and an acceptable carrier.

3. A composition of claim 2, wherein said acceptable carrier is at least one of the group consisting of plasma, blood, albumin, cell culture medium, buffer and cryopreservative;
and wherein said composition optionally further comprises at least one of a growth factor, a copper chelating agent, and a hormone.

4. The composition of claim 3, wherein said growth factor if present is G-CSF and wherein said copper chelating agent if present is D-penicillamine.

5. The composition of claim 2, wherein said composition is at a temperature sufficient to cryogenically preserve the peripheral blood stem cells.

6. The composition according to claim 2, wherein a cryopreservative is present in an amount sufficient for cryopreservation of said cells, and wherein said composition is at a temperature of from about −120° C. to about −196° C.

7. The composition according to claim 6, wherein said temperature is from about −130° C. to about −150° C.

8. The composition according to claim 2, further comprising a pharmaceutically acceptable carrier.

9. The composition according to claim 3, wherein said composition comprises a total amount of a cryopreservative selected from the group consisting of 20 to 40% dimethyl sulfoxide solution in 60 to 80% amino acid-glucose solution; 15 to 25% hydroxyethyl starch solution; 4 to 6% glycerol, 3 to 5% glucose and 6 to 10% dextran T10; 15 to 25% polyethylene glycol; and 75 to 85% amino acid-glucose solution.

10. The composition of claim 2, wherein said composition is free of toxic material.

11. A composition comprising the TVEMF-expanded peripheral blood stem cells of claim 2 wherein said acceptable carrier is at least one of the group consisting of plasma, blood, albumin, cell culture medium, buffer and cryopreservative; and wherein said composition optionally further comprises at least one of a growth factor, a copper chelating agent, and a hormone.

12. The composition of claim 11, wherein said growth factor if present is G-CSF and wherein said copper chelating agent if present is D-penicillamine.

13. The composition according to claim 11, wherein said composition further comprises a total amount of cryopreservative selected from the group consisting of 20 to 40% dimethyl sulfoxide solution in 60 to 80% amino acid-glucose solution; 15 to 25% hydroxyethyl starch solution; 4 to 6% glycerol, 3 to 5% glucose and 6 to 10% dextran T10; 15 to 25% polyethylene glycol; and 75 to 85% amino acid-glucose solution.

14. The composition of claim 11, wherein said composition is at a temperature sufficient to cryogenically preserve the peripheral blood stem cells.

15. The composition according to claim 11, wherein a cryopreservative is present and wherein said composition is at a temperature of from about −120° C. to about −196° C.

16. The composition according to claim 11, wherein said temperature is from about −130° C. to about −150° C.

17. A process for preparing a peripheral blood stem cell composition comprising the steps of:
a. placing a peripheral blood mixture comprising peripheral blood stem cells in a culture chamber of a rotatable TVEMF-bioreactor comprising a TVEMF source to establish a three-dimensional culture;
b. rotating the rotatable TVEMF-bioreactor comprising a coil about its horizontal longitudinal central axis to suspend the cells without substantial differentiation and wherein the cells are genetic expression modified according to the three-dimensional culture; and
c. subjecting cells to a TVEMF and TVEMF-expanding the peripheral blood stem cells in the TVEMF-bioreactor to prepare the peripheral blood stem cell composition.

18. The process according to claim 17, wherein said TVEMF source emits a TVEMF signal selected from the group consisting of a magnetic field amplitude of between about 10 to 100 Gauss and exhibiting a magnetic slew rate greater than 1000 Gauss per second, a magnetic field amplitude between about 0.1 to 10 Gauss along a bipolar square wave function at a frequency of between 1 to 100 Hz, a magnetic field amplitude between about 0.1 to 10 Gauss along a square wave function having a duty cycle between about 0.1 to 99.9 percent, a magnetic field having a magnetic slew rate greater than about 1000 Gauss per second that has a active duty pulse duration of less than 1 ms, a magnetic field having a magnetic slew rate greater than about 50 Gauss per second exhibiting bipolar pulses having an active duty cycle of less than 1%, a magnetic field between about 1 to 100 Gauss peak-to-peak and having a magnetic slew rate bipolar pulses with an active duty cycle of less than 1%, and a time-dependent magnetic field exhibiting a relatively uniform magnetic field strength throughout the cell mixture contents.

19. The process according to claim 17, wherein the TVEMF-expanding step continues until the number of TVEMF-expanded peripheral blood stem cells is more than 7 times the number of peripheral blood stem cells placed in the TVEMF-bioreactor.

20. The process according to claim 17, further comprising collecting peripheral blood prior to placing the peripheral blood mixture in a TVEMF-bioreactor.

21. The process of claim 20, wherein said peripheral blood is human peripheral blood.

22. The process according to claim 17, further comprising collecting thawed cryopreserved peripheral blood from a peripheral blood storage facility prior to adding the peripheral blood to the peripheral blood mixture.

23. The process of claim 17, further comprising a step of removing toxic material from the peripheral blood mixture prior to TVEMF-expansion.

24. The process of claim 17, wherein the TVEMF-bioreactor has an integral TVEMF source.

25. The process of claim 17, wherein the TVEMF-bioreactor has an adjacent TVEMF source.

26. The process of claim 17, further comprising the steps of transferring the TVEMF-expanded cells of the peripheral blood stem cell composition into a cryogenic container having a temperature, and lowering the temperature of the cryogenic container to a temperature of from −120° C. to −196° C. at a controlled rate.

27. A composition comprising peripheral blood stem cells and an acceptable carrier prepared by the process according to claim 17.

28. A method of treating a disease of a mammal comprising the step of administering to the mammal a therapeutically effective amount of a composition comprising the peripheral blood stem cells of claim 1 and a pharmaceutically acceptable carrier.

29. A method of treating a disease of a mammal comprising the step of administering to the mammal a therapeutically effective amount of a composition comprising the TVEMF-expanded peripheral blood stem cells of claim 17 and a pharmaceutically acceptable carrier.

30. The method of claim 1 or 28, wherein the mammal is a human and wherein the mammal is the source of the peripheral blood stem cells prior to TVEMF-expansion.

31. The method of claim 28 or 29, wherein the amount of TVEMF-expanded peripheral blood stem cells to be administered to the mammal is at least 20 ml of a composition having 10⁷ to 10⁹ stem cells/ml.

32. The method of claim 29, wherein the disease is selected from at least one of the group consisting of diseases resulting from a failure or dysfunction of normal blood cell production and maturation, hyperproliferative stem cell disorders, aplastic anemia, pancytopenia, thrombocytopenia, red cell aplasia, Blackfan-Diamond syndrome due to drugs, radiation, or infection, idiopathic; hematopoietic malignancies, acute lymphoblastic (lymphocytic) leukemia, chronic lymphocytic leukemia, acute myelogenous leukemia, chronic myelogenous leukemia, acute malignant myelosclerosis, multiple myeloma, polycythemia vera, agnogenic myelometaplasia, Waldenstrom's macroglobulinemia, Hodgkin's lymphoma, non-Hodgkins's lymphoma; immunosuppression in patients with malignant, solid tumors, malignant melanoma, carcinoma of the stomach, ovarian carcinoma, breast carcinoma, small cell lung, carcinoma, retinoblastoma, testicular carcinoma, glioblastoma, rhabdomyosarcoma, neuroblastoma, Ewing's sarcoma, lymphoma; autoimmune diseases, rheumatoid arthritis, diabetes type I, chronic hepatitis, multiple sclerosis, and systemic lupus erythematosus; genetic (congenital) disorders, anemias, familial aplastic, Fanconi's syndrome, Bloom's syndrome, pure red cell aplasia (PRCA), dyskeratosis congenital, Blackfan-Diamond syndrome, congenital dyserythropoietic syndromes I-IV, Chwachmann-Diamond syndrome, dihydrofolate reductase deficiencies, formamino transferase deficiency, Lesch-Nyhan syndrome, congenital spherocytosis, congenital elliptocytosis, congenital stomatocytosis, congenital Rh null disease, paroxysmal nocturnal hemoglobinuria, G6PD (glucose-6-phosphate dehydrogenase), variants 1,2,3, pyruvate kinase deficiency, congenital erythropoietin sensitivity, deficiency, sickle cell disease and trait, thalassemia alpha, beta, gamma met-hemoglobinemia, congenital disorders of immunity, severe combined immunodeficiency disease, (SCID), bare lymphocyte syndrome, ionophore-responsive combined, immunodeficiency, combined immunodeficiency with a capping abnormality, nucleoside phosphorylase deficiency, granulocyte actin deficiency, infantile agranulocytosis, Gaucher's disease, adenosine deaminase deficiency, Kostmann's syndrome, reticular dysgenesis, congenital leukocyte dysfunction syndromes; osteopetrosis, myelosclerosis, acquired hemolytic anemias, acquired immunodeficiencies, infectious disorders causing primary or secondary immunodeficiencies, bacterial infections (e.g., Brucellosis, Listerosis, tuberculosis, leprosy), parasitic infections (e.g., malaria, Leishmaniasis), fungal infections, disorders involving disproportions in lymphoid cell sets and impaired immune functions due to aging phagocyte disorders, Kostmann's agranulocytosis, chronic granulomatous disease, Chediak-Higachi syndrome, neutrophil actin deficiency, neutrophil membrane GP-180 deficiency, metabolic storage diseases, mucopolysaccharidoses, mucolipidoses, miscellaneous disorders involving immune mechanisms, Wiskott-Aldrich Syndrome, and alpha 1-antitrypsin deficiency.

33. A method of researching a disease state comprising introducing a TVEMF-expanded stem cell into a test system for the disease state.

34. The method of claim 33 wherein said disease state is at least one of the group consisting of diseases resulting from a failure or dysfunction of normal blood cell production and maturation, hyperproliferative stem cell disorders, aplastic anemia, pancytopenia, thrombocytopenia, red cell aplasia, Blackfan-Diamond syndrome due to drugs, radiation, or infection, idiopathic; hematopoietic malignancies, acute lymphoblastic (lymphocytic) leukemia, chronic lymphocytic leukemia, acute myelogenous leukemia, chronic myelogenous leukemia, acute malignant myelosclerosis, multiple myeloma, polycythemia vera, agnogenic myelometaplasia, Waldenstrom's macroglobulinemia, Hodgkin's lymphoma, non-Hodgkins's lymphoma; immunosuppression in patients with malignant, solid tumors, malignant melanoma, carcinoma of the stomach, ovarian carcinoma, breast carcinoma, small cell lung, carcinoma, retinoblastoma, testicular carcinoma, glioblastoma, rhabdomyosarcoma, neuroblastoma, Ewing's sarcoma, lymphoma; autoimmune diseases, rheumatoid arthritis, diabetes type I, chronic hepatitis, multiple sclerosis, and systemic lupus erythematosus; genetic (congenital) disorders, anemias, familial aplastic, Fanconi's syndrome, Bloom's syndrome, pure red cell aplasia (PRCA), dyskeratosis congenital, Blackfan-Diamond syndrome, congenital dyserythropoietic syndromes I-IV, Chwachmann-Diamond syndrome, dihydrofolate reductase deficiencies, formamino transferase deficiency, Lesch-Nyhan syndrome, congenital spherocytosis, congenital elliptocytosis, congenital stomatocytosis, congenital Rh null disease, paroxysmal nocturnal hemoglobinuria, G6PD (glucose-6-phosphate dehydrogenase), variants 1,2,3, pyruvate kinase deficiency, congenital erythropoietin sensitivity, deficiency, sickle cell disease and trait, thalassemia alpha, beta, gamma met-hemoglobinemia, congenital disorders of immunity, severe combined immunodeficiency disease, (SCID), bare lymphocyte syndrome, ionophore-responsive combined, immunodeficiency, combined immunodeficiency with a capping abnormality, nucleoside phosphorylase deficiency, granulocyte actin deficiency, infantile agranulocytosis, Gaucher's disease, adenosine deaminase deficiency, Kostmann's syndrome, reticular dysgenesis, congenital leukocyte dysfunction syndromes; osteopetrosis, myelosclerosis, acquired hemolytic anemias, acquired immunodeficiencies, infectious disorders causing primary or secondary immunodeficiencies, bacterial infections (e.g., Brucellosis, Listerosis, tuberculosis, leprosy), parasitic infections (e.g., malaria, Leishmaniasis), fungal infections, disorders involving disproportions in lymphoid cell sets and impaired immune functions due to aging phagocyte disorders, Kostmann's agranulocytosis, chronic granulomatous disease, Chediak-Higachi syndrome, neutrophil actin deficiency, neutrophil membrane GP-180 deficiency, metabolic storage diseases, mucopolysaccharidoses, mucolipidoses, miscellaneous disorders involving immune mechanisms, Wiskott-Aldrich Syndrome, and alpha 1-antitrypsin deficiency.

35. Bone marrow stem cells from a mammal, wherein said bone marrow stem cells are TVEMF-expanded.

36. The process of claim 17 wherein the peripheral blood stem cells are expanded to two times the number that were placed in the culture chamber.

37. A process for preparing an expanded peripheral blood stem cell composition comprising the steps of:
placing a peripheral blood stem cell mixture comprising peripheral blood stem cells in a rotatable bioreactor;
expanding the peripheral blood stem cells by rotating the bioreactor about a substantially horizontal vertical central axis to suspend the cells in the rotating bioreactor;
preparing an expanded peripheral blood stem cell composition with the expanded peripheral blood stem cells.

38. The method of claim 37 wherein the number of expanded cells is less than the number that were placed in the TVEMF-bioreactor.

39. The method of claim 37 wherein the number of expanded cells is at least one more than the number that were placed in the TVEMF-bioreactor.

40. The method of claim 37 wherein the number of expanded cells is the same as the number placed in the TVEMF-bioreactor.

41. The method of claim 37 further comprising the step of continuing expanding the cells until the cells are expanded to at least seven times the number that were placed in the rotatable bioreactor.

42. An expanded peripheral blood stem cell composition prepared by the process claimed in claim 37 further comprising a pharmaceutically acceptable carrier.

43. A method of treating a disease of a mammal comprising the step of administering to the mammal a therapeutically effective amount of the composition as in claim 42.

44. The method of claim 43, wherein the mammal is a human and wherein the mammal is the source of the peripheral blood stem cells prior to TVEMF-expansion.

45. The method of claim 43, wherein the amount of TVEMF-expanded peripheral blood stem cells to be administered to the mammal is at least 20 ml of a composition having 10⁷ to 10⁹ stem cells/ml.
